(19) United States
(12) Patent Application Publication
(10) Pub. No.: US 2025/0381304 A1
(43) Pub. Date: Dec. 18, 2025

(54) METHODS AND COMPOSITIONS FOR IN VIVO EDITING OF STEM CELLS

(71) Applicant: CRISPR THERAPEUTICS AG, Zug (CH)

(72) Inventors: Kyungah MAENG, South Boston, MA (US); Seshidhar Reddy POLICE, South Boston, MA (US)

(21) Appl. No.: 18/877,165

(22) PCT Filed: Jun. 21, 2023
(86) PCT No.: PCT/IB2023/056404

§ 371 Date: Dec. 19, 2024

Related U.S. Application Data
(60) Provisional application No. 63/354,181, filed on Jun. 21, 2022.

(51) Int. Cl. A61K 48/00; C12N 9/22; C12N 15/11; C12N 15/86

(52) U.S. Cl. CPC A61K 48/0058; C12N 9/226; C12N 15/11; C12N 15/86; C12N 2310/20; C12N 2750/14143

(57) ABSTRACT

The present disclosure includes methods, vectors, compositions and kits for in vivo editing of stem cells in a subject.

### CROSS-REFERENCE TO RELATED APPLICATIONS

This application is a U.S. national phase application under 35 U.S.C. § 371 of International Application No. PCT/IB2023/056404, filed on Jun. 21, 2023 and published as WO 2023/248147 A2 on Dec. 28, 2023, which claims the benefit under 35 U.S.C. § 119(e) to U.S. Provisional Patent Application No. 63/354,181, filed on Jun. 21, 2022; the content of each of these related applications is incorporated herein by reference in its entirety for all purposes.

### REFERENCE TO SEQUENCE LISTING

The present application is being filed along with a Sequence Listing in electronic format. The Sequence Listing is provided as a file entitled 80EM-341713-US_SeqList, created Jun. 5, 2023, which is 63 kilobytes in size. The information in the electronic format of the Sequence Listing is incorporated herein by reference in its entirety.

### BACKGROUND

### Field

The present disclosure generally relates to the field of molecular biology and biotechnology, including gene editing.

### Description of the Related Art

The targeting of DNA using the RNA-guided, DNA-targeting principle of CRISPR (Clustered Regularly Interspaced Short Palindromic Repeats)-Cas (CRISPR associated) systems has been widely used. CRISPR-Cas systems can be divided in two classes, with class 1 systems utilizing a complex of multiple Cas proteins (such as type I, III, and IV CRISPR-Cas systems) and class 2 systems utilizing a single Cas protein (such as type II, V, and VI CRISPR-Cas systems). Type II CRISPR-Cas-based systems have been used for genome editing, and require a Cas polypeptide or variant thereof guided by a customizable guide RNA (gRNA) for programmable DNA targeting.

Methods of genetically editing stem cells ex vivo have been developed. However, ex vivo stem cell editing is a complex, lengthy and multi-step process that often requires life-threatening conditioning regimen and can only be performed in hospitals equipped with stem cell transplantation facilities.

There still remains a need for developing simple and effective gene therapy to genetically modify stem cells, e.g., genetic modification in vivo.

### SUMMARY

Disclosed herein include methods, compositions, and kits for in vivo editing hematopoietic stem cells (HSCs) and/or hematopoietic progenitor cells (HPCs).

Disclosed herein include a method for in vivo editing HSCs and/or HPCs in a subject in need thereof. The method, in some embodiments, comprises administering to the subject a mobilization composition capable of mobilizing the HSCs and/or HPCs in the subject; and administering to the subject a plurality of adeno-associated virus 9 (AAV9) vectors encapsulating (a) at least one guide RNA (gRNA) that targets a genomic region of interest or a nucleic acid encoding the at least one gRNA, and (b) a nucleic acid encoding a RNA-guided endonuclease, thereby editing the HSCs and/or HPCs in the subject. The method can further comprises administering to the subject a second mobilization composition capable of mobilizing HSC and/or HPC in the subject after administering a first mobilization composition to the subject. In some embodiments, the subject is administered with the first mobilization composition and/or the second mobilization composition daily for two, three, four, five, six, seven, or eight consecutive days. In some embodiments, the first mobilization composition is different from the second mobilization composition. In some embodiments, the first mobilization composition is administered to the subject about one hour to about six hours before the administration of the second mobilization composition. In some embodiments, the first mobilization composition and/or the second composition comprises a mobilization agent selected from the group consisting of plerixafor or an analog or derivative thereof, granulocyte colony-stimulating factor (G-CSF) or an analog or derivatives thereof, GRO-β or an analog or derivative thereof, granulocyte macrophage colony stimulating factor (GM-CSF) or an analog or derivative thereof, stem cell factor or an analog or derivative thereof, a modulator of SDF-1/CXCR4 axis, a sphingosine-1-phosphate (S1P) agonist, a VCAM/VLA4 inhibitor, parathyroid hormone (PTH) or an analog or derivative thereof, a proteosome inhibitor, and a combination thereof. In some embodiments, the mobilization agent is administered to the subject in an amount of about 0.1-20 mg/kg of the subject per administration. In some embodiments, the first mobilization composition comprises plerixafor, and the second mobilization composition comprises plerixafor and G-CSF. In some embodiments, the subject is administered with the plurality of AAV9 vectors once, two times, or three times. In some embodiments, the plurality of AAV9 vectors is administered to the subject after the administration of the first and/or the mobilization composition, and optionally at least about 0.5 hour, 1 hour, 1.5 hours, 2 hours, 3 hours, or 4 hours after the administration of the first and/or the mobilization composition. In some embodiments, the plurality of AAVs is administered to the subject at a dose of about 1E14 v/kg to 5E14 vg/kg per administration.

The method, in some embodiments, comprises identifying a subject in need of the administration. In some embodiments, the genomic region of interest comprises a gene of interest. In some embodiments, the gene of interest is B-cell lymphoma/leukemia 11A (BCL11A) gene and the subject is a subject having a β-thalassemia and sickle cell disease. In some embodiments, at least one of the plurality of AAV9 vectors comprises a nucleic acid encoding the RNA-guided endonuclease and the gRNA that targets BCL11A gene. In some embodiments, the nucleic acid encoding a RNA-guided endonuclease is a mRNA of the RNA-guided endonuclease. The RNA-guided endonuclease can be a Cas9 endonuclease, for example, S. pyogenes Cas9, S. aureus Cas9, N. meningitides Cas9, S. thermophilus Cas9, S. thermophilus 3 Cas9, T. denticola Cas9, or a variant thereof. In some embodiments, the at least one gRNA is a single-guide RNA (sgRNA). In some embodiments, (a) the at least one gRNA or the nucleic acid encoding the at least one gRNA, and (b) the nucleic acid encoding the RNA-guided nuclease are encapsulated in a same AAV9. In some embodiments, (a) the at least one gRNA or the nucleic acid encoding the at least one gRNA, and (b) the nucleic acid encoding the RNA-guided nuclease are encapsulated in separate AAV9 vectors. In some embodiments, the plurality of AAV9 vectors are HSC-tropic. The subject can be human. The plurality of AAV9 vectors can be administered to the subject via, for example, intravenous administration or systemic administration.

Disclosed herein includes an adeno-associated virus (AAV) vector. The AAV vector can comprise, for example, an AAV9 capsid encapsulating (a) one or two guide RNAs (gRNAs) that targets a genomic region of interest, or a nucleic acid encoding the one or two gRNAs; and (b) a nucleic acid encoding a RNA-guided endonuclease, wherein the genomic region of interest comprises a gene of interest that is preferentially expressed in hematopoietic cells. Disclosed herein includes a composition, in some embodiments, comprises a first AAV vector comprising an AAV9 capsid encapsulating one or two guide RNAs (gRNAs) that target a genomic region of interest or a nucleic acid encoding the one or two gRNAs; and a second AAV vector comprising an AAV9 capsid encapsulating a nucleic acid encoding a RNA-guided endonuclease, wherein the genomic region of interest comprises a gene of interest that is preferentially expressed in hematopoietic cells. In some embodiments, the gene of interest is a gene that is preferentially expressed in HSCs and/or HPCs. In some embodiments, the nucleic acid encoding a RNA-guided endonuclease is a mRNA of the RNA-guided endonuclease. In some embodiments, the RNA-guided endonuclease is a Cas9 endonuclease, including but not limited to, S. pyogenes Cas9 (SpCas9), S. aureus Cas9 (SaCas9), S. lugdunensis Cas9 (SluCas9), N. meningitidis Cas9, S. thermophilus Cas9, S. thermophilus 3 Cas9, T. denticola Cas9, C. jejuni Cas9 (CjCas9), or a variant thereof. In some embodiments, at least one of the one or two gRNAs is a single-guide RNA (sgRNA). In some embodiments, at least one of the one or two gRNAs targets the erythroid specific enhancer of BCL11A gene.

Also disclosed herein includes a pharmaceutical composition, comprising any one or more of the AAV vectors or compositions disclosed herein.

### BRIEF DESCRIPTION OF THE DRAWINGS

FIG. 1 depicts a non-limiting exemplary in vivo hematopoietic stem cell (HSC) editing platform.

FIG. 2 depicts a non-limiting exemplary workflow of in vivo HSC editing.

FIG. 3A-FIG. 3D illustrate screening and selection of human HSC tropic adeno-associated virus (AAV) vectors.

FIG. 4 illustrates screening and selection of SaCas9 gRNAs for disruption of human C-C chemokine receptor type 5 (CCR5) gene. Top panel illustrates a non-limiting exemplary SaCas9 all-in-one vector comprising SaCas9 nucleic acid and the gRNA targeting exon 3 of CCR5. Bottom panel shows the editing efficiencies of two gRNAs, T10 and T13, in HSCs.

FIG. 5 illustrates an exemplary animal study to identify optimal HSC mobilization and AAV vector dosing regimen.

FIG. 6 illustrates an exemplary animal study to identify optimal HSC mobilization and AAV vector dosing regimen.

FIG. 7 illustrates screening and selection of optimal HSC mobilization regimen.

FIG. 8A illustrates a non-limiting exemplary regimen of in vivo editing of HSCs in humanized mice. FIG. 8B illustrates a non-limiting exemplary all-in-two vector with one vector (AAV9/CTX-462) comprising a nucleic acid encoding SpCas9 and the other vector (AAV9/CTX-1419: U6-TB7+U6-TB50) comprising SpCas9 gRNA TB7 and SpCas9 gRNA TB50. FIG. 8C shows CCR5 editing efficiency of the all-in-two vector in CD34⁺ cells in comparison to a PBS control.

FIG. 9A illustrates a non-limiting exemplary regimen of in vivo editing of HSCs in humanized mice. FIG. 9B-FIG. 9D show relative CCR5 mRNA expression level in splenocytes (FIG. 9B), CD4⁺ T cells (FIG. 9C) and B cells (FIG. 9D) of the humanized mice treated with AAV9/CTX-1419 and a PBS control.

FIG. 10A illustrates a non-limiting exemplary regimen of in vivo editing of HSCs in humanized mice following a primary engraftment. FIG. 10B is a plot showing CCR5 editing efficiency of the AAV9-CTX-1419 vector in the CD34⁺ population with bi-allelic editing and mono-allelic editing. FIG. 10C is a plot showing CCR5 editing efficiency by the AAV9-CTX-1419 vector in comparison to a PBS control obtained from HSC cluster analysis.

FIG. 10D illustrates a non-limiting exemplary regimen of in vivo HSC editing in humanized mice following a secondary engraftment. FIG. 10E is a plot showing CCR5 editing frequency after the secondary engraftment.

FIG. 11 is a graph showing the results of in vivo editing of HSCs with AAV. Additionally, preservation of editing in secondary engraftment studies confirmed editing of true long-term HSCs.

FIG. 12A-FIG. 12D illustrate screening, location, and off-target analysis of exemplary ccr5-targeting gRNAs. FIG. 12A illustrates a diagram of the screening process used to identify optimal gRNAs from the initial pool of 123 gRNAs predicted to have double stranded breaks in ccr5 exon 3. FIG. 12B illustrates a genome organization of the ccr5 locus, and the relative positions of each of the 4 optimal gRNAs within the ccr5 exon 3 open reading frame and in relation to the naturally occurring Δ32 deletion. FIG. 12C-FIG. 12D illustrate indel frequency of exemplary gRNAs. CD34⁺ HSCs were edited with each lead gRNA using a concentration of 150 μg/mL or mock edited. Predicted off-target sites were amplified, sequenced, an analyzed for indel formation. The blue bars represent indel frequency at the off-target sites for gRNAs TB8 (FIG. 12C), TB48 (FIG. 12D), TB7 (FIG. 12D), and TB50 (FIG. 12D) in unedited CD34⁺ HSCs. The orange bars represent indel frequency at off-target sites in CD34⁺ HSCs treated with the gRNAs and SpCas9 protein. An indel frequency level of >0.10%, represented by the dashed line, was considered the threshold for off-target editing. The off-target sequences are listed in Table 4.

FIG. 13A-FIG. 13B illustrate editing efficiency of exemplary ccr5-targeting gRNAs in primary human HSCs. Of the 123 gRNA sequences identified by the in silico predictor software, 15 were excluded for having multiple target sites within the human genome. The remaining 108 gRNAs were produced through in vitro transcription and screened for editing efficiency in two CD34⁺ HSC donors. FIG. 13A depicts the average indel frequency of the two HSC donors for each of the 108 guides. FIG. 13B depicts the 11 guides demonstrating the highest editing efficiency and without homology to the ccr2 gene which were chemically synthesized in an optimized format and electroporated at 37.5, 75, or 150 μg/mL concentrations using two CD34⁺ HSC donor sources. All bars represent the mean, and all errors bars represent SD.

FIG. 14 depicts efficient disruption of ccr5 gene in primary human T cells by exemplary single ccr5-targeting gRNAs (TB7-brown, TB8-pink, TB48-blue, TB50-red), or a dual gRNA approach (TB48+TB50, purple).

FIG. 15A-FIG. 15B depict ccr5 gene disruption efficiency of exemplary ccr5-targeting gRNAs (FIG. 15A) and number and type of colonies derived from single HSCs treated with ccr5-targeting gRNAs compared to controls (FIG. 15B). FIG. 15A: Mobilized CD34⁺ HSCs derived from three healthy adult donors were electroporated with Cas9 complexed to gRNAs TB48 and TB50. Single gRNA gene disruptions were measured by Sanger sequencing of ccr5 amplified from genomic DNA isolated 48-hours after electroporation. Dual gRNA lesions, labeled as “Deletion”, were measured via droplet-digital PCR. FIG. 15B: Number and type of colonies derived from single HSCs from each condition (culture control, ccr5-edited and GFP gRNA mock edited) cultured in a methylcellulose-based colony forming unit assay. The results demonstrate that high-efficiency gene disruption in human HSCs does not significantly alter engraftment potential or hematopoiesis.

FIG. 16 illustrates a BCL11A gene structure and locations of gRNA binding sites in the erythroid specific enhancer of the BCL11A gene.

FIG. 17 is a plot showing gene editing efficiency by exemplary guide RNAs SaG1, SluG1, SluG2, SluG3 and SluG4 targeting erythroid DNase I hypersensitive sites (DHS) +55 and +58 of the BCL11A gene.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include an in vivo method for editing stem cells (e.g., hematopoietic stem cells). The method comprises administering to the subject a mobilization agent in an effective amount to produce mobilized stem cells and administering to the subject a plurality of AAVs encapsulating (a) at least one guide RNA (gRNA) or a nucleic acid encoding the at least one gRNA that targets a gene of interest, and (b) a nucleic acid encoding a RNA-guided endonuclease, thereby editing the gene of interest in the subject.

### Definition

As used herein, the term “about” means plus or minus 5% of the provided value.

As used herein, the term “RNA-guided endonuclease” refers to a polypeptide capable of binding a RNA (e.g., a gRNA) to form a complex targeted to a specific DNA sequence (e.g., in a target DNA). A non-limiting example of RNA-guided endonuclease is a Cas polypeptide (e.g., a Cas endonuclease, such as a Cas9 endonuclease). In some embodiments, the RNA-guided endonuclease as described herein is targeted to a specific DNA sequence in a target DNA by an RNA molecule to which it is bound. The RNA molecule can include a sequence that is complementary to and capable of hybridizing with a target sequence within the target DNA, thus allowing for targeting of the bound polypeptide to a specific location within the target DNA.

As used herein, the term “guide RNA” or “gRNA” refers to a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

As used herein, a “secondary structure” of a nucleic acid molecule (e.g., an RNA fragment, or a gRNA) refers to the base pairing interactions within the nucleic acid molecule.

As used herein, the term “target DNA” refers to a DNA that includes a “target site” or “target sequence.” The term “target sequence” is used herein to refer to a nucleic acid sequence present in a target DNA to which a DNA-targeting sequence or segment (also referred to herein as a “spacer”) of a gRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a gRNA and the target sequence can, for example, be based on Watson-Crick base pairing rules, which enables programmability in the DNA-targeting sequence or segment. The DNA-targeting sequence or segment of a gRNA can be designed, for instance, to hybridize with any target sequence.

As used herein, the term “Cas endonuclease” or “Cas nuclease” refers to an RNA-guided DNA endonuclease associated with the CRISPR adaptive immunity system.

Unless otherwise indicated “nuclease” and “endonuclease” are used interchangeably herein to refer to an enzyme which possesses endonucleolytic catalytic activity for polynucleotide cleavage.

As used herein, the term “invariable region” of a gRNA refers to the nucleotide sequence of the gRNA that associates with the RNA-guided endonuclease. In some embodiments, the gRNA comprises a crRNA and a transactivating crRNA (tracrRNA), wherein the crRNA and tracrRNA hybridize to each other to form a duplex. In some embodiments, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some embodiments, the invariable region of the gRNA refers to the portion of the crRNA that is the minimum CRISPR repeat sequence and the tracrRNA.

As used herein, the term “donor template” refers to a nucleic acid strand containing exogenous genetic material which can be introduced into a genome (e.g., by a homology directed repair) to result in targeted integration of the exogenous genetic material. In some embodiments, a donor template can have no regions of homology to the targeted location in the DNA and can be integrated by NHEJ-dependent end joining following cleavage at the target site. A donor template can be DNA or RNA, single-stranded or double-stranded, and can be introduced into a cell in linear or circular form.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases. The nucleic acid can be composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹M, 10⁻¹⁰ M, 10⁻¹¹ M, 10⁻¹²M, 10⁻¹³ M, 10⁻¹⁴ M, 10⁻¹⁵M, or a number or a range between any two of these values. Kd can be dependent on environmental conditions, e.g., pH and temperature. “Affinity” refers to the strength of binding, and increased binding affinity is correlated with a lower Kd.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. In some embodiments, a splint oligonucleotide sequence is not more than about 50% identical to one of the two polynucleotides (e.g., RNA fragments) to which it is designed to be complementary. The complementary portion of each sequence can be referred to herein as a ‘segment’, and the segments are substantially complementary if they have 80% or greater identity.

The terms “complementarity” and “complementary” mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g. complete complementarity) or imperfect (e.g. partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e. 100%. For example, the complementary candidate sequence segment is perfectly complementary to the candidate sequence segment, whose sequence can be deducted from the candidate sequence segment using the Watson-Crick base pairing rules.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

The term “AAV” or “adeno-associated virus” refers to a Dependoparvovirus within the Parvoviridae genus of viruses.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the terms “transfection” or “infection” refer to the introduction of a nucleic acid into a host cell, such as by contacting the cell with a recombinant MVA virus or a gutless picornaviral particle as described herein.

As used herein, the term “transgene” refers to any nucleotide or DNA sequence that is integrated into one or more chromosomes of a target cell by human intervention. In some embodiment, the transgene comprises a polynucleotide that encodes a protein of interest. The protein-encoding polynucleotide is generally operatively linked to other sequences that are useful for obtaining the desired expression of the gene of interest, such as transcriptional regulatory sequences. In some embodiments, the transgene can additionally comprise a nucleic acid or other molecule(s) that is used to mark the chromosome where it has integrated.

As used herein, the terms “hematopoietic progenitor cell” (HPC) and “hematopoietic stem cell” (HSC) refer to cells of a stem cell lineage that give rise to all the blood cell types, for example erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells).

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjuster controller, isotonic agent and other conventional additives may also be added to the carriers.

As used herein, the terms “effective amount” or “pharmaceutically effective amount” or “therapeutically effective amount” refer to an amount sufficient to effect beneficial or desirable biological and/or clinical results.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipient can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. I some embodiments, the subject is a mammal. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a primate. In some embodiments, the mammal is a human. In some embodiments, the mammal is not a human.

Ex vivo based stem cell therapy is a complex, lengthy and multi-step process involving cell isolation and transplantation which can only be done in hospitals equipped with stem cell transplantation facilities and often requires life-threatening conditioning regiment. There remains needs for developing a safe, simple, rapid and more affordable gene therapy which can be used to treat a number of patients who share the same or similar genotype or allele.

The present disclosure provides simple, affordable and highly efficient in vivo gene editing method in stem cells and related vectors, compositions and kits. The methods and related vectors, compositions and kits can directly target a gene of interest or variants thereof and permanently reduce the expression, function, or activity of the gene. In some embodiments, the vectors, methods, compositions, and kits described herein can reduce the expression level of the gene in the hematopoietic stem cells of a subject under treatment by at least 50% or greater. The methods and related vectors, compositions and kits used herein can also significantly minimize the number and frequency of off-target effects, thus reducing the risk of genotoxicity.

### In Vivo Editing of Stem Cells

Provided herein includes vectors, compositions and methods for in vivo editing of stem cells by functionally knocking out or reducing the expression of a gene of interest in the genome of a stem cell in a subject (e.g., a human). The in vivo editing approach described herein edits the chromosomal DNA of the cells in a patient using the vectors and compositions herein described. The cells can be stem cells, bone marrow cells, hematopoietic stem cells and/or other B and T cell progenitors, such as CD34⁺ cells. In vivo treatment can eliminate problems and losses associated with ex vivo treatment and engraftment. The in vivo treatment disclosed herein is a simple and rapid process in which one vector system (e.g., AAV vector) can be used to treat more than one patient, for example a number of patients who share the same or similar genotype or allele. Compared to ex vivo treatment, the in vivo treatment does not require risky conditioning regimen, can be performed in outpatient settings, and is less expensive for more patients worldwide.

In some embodiments, a method for in vivo editing a stem cell in a subject in need thereof comprises administering to the subject a mobilization agent in an effective amount to produce mobilized stem cells and administering to the subject a plurality of AAVs encapsulating (a) at least one guide RNA (gRNA) or a nucleic acid encoding the at least one gRNA that targets a genomic region of interest (e.g., a gene of interest), and (b) a nucleic acid encoding a RNA-guided endonuclease, thereby editing the stem cell in the subject. The gene of interest can be, for example, a gene that is preferentially expressed in hematopoietic cells, for example hematopoietic stem cells (HSCs) and/or hematopoietic progenitor cells (HPCs). Non-limiting examples of gene that is preferentially expressed in hematopoietic cells include BCL11A, OCT6 and GATA-2. In some embodiments, the gene of interest is BCL11A.

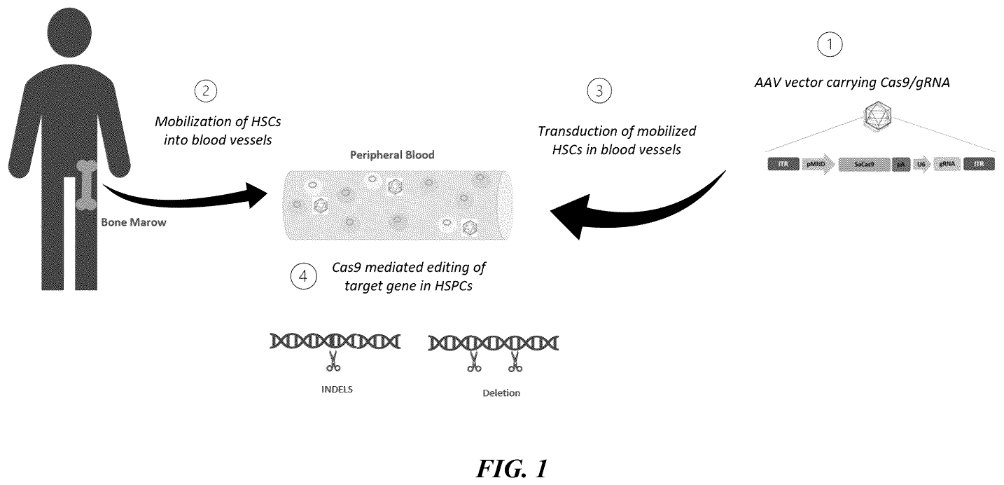

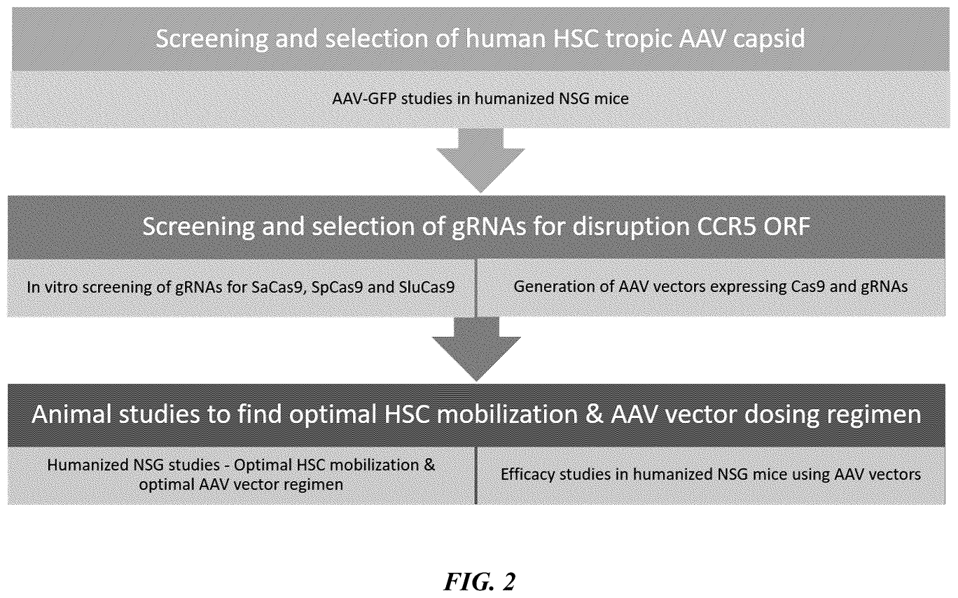

The mobilization agent administered to the subject mobilizes stem cells into blood vessels of the subject. The mobilized stem cells can then be transduced with the plurality of viral particles (e.g., AAVs) carrying the one or more nucleic acids herein described (e.g., at least one gRNA targeting a gene of interest and a nucleic acid encoding a DNA endonuclease), thereby editing the gene of interest in the stem cells of the subject. An exemplary schematic of the method is shown in FIG. 1.

The term “mobilizing” as used herein with reference to stem cells refers to the act of migrating the stem cells (e.g., hematopoietic stem cells) from a first location (e.g., bone marrow) into a second location (e.g., peripheral blood). Mobilizing the stem cells can be performed by administering to the subject in need an effective amount of a mobilization agent. The term “mobilization agent” refers to a drug used to cause the movement of stem cells from the bone marrow into the peripheral blood. In some embodiments, the mobilization agent comprises a CXCR4 antagonist (e.g., plerixafor or analogs or derivatives thereof) that can block the CXCR4 receptor and prevent its activation. In some embodiments, the mobilization agent comprises granulocyte colony stimulating factor (G-CSF) and glycosylated or pegylated forms thereof. Exemplary types of G-CSF include, but are not limited to, lenograstim (Granocyte), filgrastim (Neupogen, Zarzio, Nivestim, Accofil), long acting (pegylated) filgrastim (pegfilgrastim, Neulasta, Pelmeg, Ziextenco) and lipegfilgrastim (Lonquex).

In some embodiments, the mobilization agent comprises plerixafor and analogs or derivatives thereof, G-CSF or analogs or derivatives thereof, or a combination thereof. Exemplary analogs of Plerixafor include, but are not limited to, AMD11070, AMD3465, KRH-3955, T-140, and 4F-benzyol-TN14003, as described by De Clercq, E. (Pharmacol Ther. 2010 128(3): 509-18) which is incorporated by reference herein in its entirety. Non-limiting examples of mobilization agent include plerixafor or an analog or derivative thereof, granulocyte colony-stimulating factor (G-CSF) or an analog or derivatives thereof, GRO-0 or an analog or derivative thereof, granulocyte macrophage colony stimulating factor (GM-CSF) or an analog or derivative thereof, stem cell factor or an analog or derivative thereof, a modulator of SDF-1/CXCR4 axis, a sphingosine-1-phosphate (S1P) agonist, a VCAM/VLA4 inhibitor, parathyroid hormone (PTH) or an analog or derivative thereof, a proteosome inhibitor, and any combination thereof. In some embodiments, the mobilization agents comprise a combination of plerixafor and G-CSF. The combination, in some embodiments, results in enhanced stem cell mobilization and improved CCR5 editing efficiency (see e.g., Example 3). In some embodiments, the population of CD34⁺ and/or CD45⁺ cells are substantially enriched (e.g., about, at least, or at least about 5%, 10%, 15%, 20%, 30%, 40%, 50%, 60%, 70%, or more) in a subject administered with a combination of plerixafor and G-CSF.

The subject can be administered with the mobilization agent(s) one, two, three, four, five, six, seven, eight or more times for the treatment. Two administration of the mobilization agent to the subject can be consecutive or separated by a suitable time period. In some embodiments, the subject is administered with one or more mobilization agent(s) (e.g., plerixafor, G-CSF, or a combination of both) daily for two, three, four, five, six, seven, eight or more consecutive days. The mobilization agents used in the two or more administrations can be the same or different. In some embodiments, the mobilization agent(s) is administered to the subject at a dose of about 0.1-20 mg/kg, for example 1-10 mg/kg, per administration. some embodiments, the mobilization agent(s) is administered to the subject at a dose of about 0.1-20 mg/kg, for example 1-10 mg/kg, per administration. For example, plerixafor can be provided at a dose of about 1-10 mg/kg (including 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or a number or range between any two of these values), for example 3-6 mg/kg (e.g., 5 mg/kg). G-CSF can be provided at a dose of about 0.1-2 mg/kg (including 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, or a number or range between any two of these values), for example 0.1-0.5 mg/kg (e.g., 0.125 mg/kg). The dose can be the same or different for each of the two or more administrations to the subject. When two or more mobilization agents are administered to a subject, the dose for each mobilization agent can be the same or different.

The mobilized stem cells can comprise hematopoietic stem cells. In some embodiments, the hematopoietic stem cells comprise CD34⁺ peripheral blood stem cells.

The terms “stem cell” and “progenitor cell” refer to a cell with the capacity or potential, under particular circumstances, to differentiate to a more specialized or differentiated phenotype, and which retains the capacity, under certain circumstances, to proliferate without substantially differentiating. In some embodiments, the progenitor cell or stem cell refers to a generalized mother cell whose descendants (progeny) specialize, by differentiation, for example, by acquiring completely individual characters as occurs in progressive diversification of embryonic cells and tissues.

A “differentiated cell” is a cell that has progressed further down the developmental pathway than the cell to which it is being compared. A differentiated cell may derive from a multipotent cell that itself is derived from a multipotent cell, and so on. Thus, stem cells can differentiate into lineage-restricted precursor cells (such as a hematopoietic progenitor cell), which in turn can differentiate into other types of precursor cells further down the pathway (such as a hematopoietic precursor), and then to an end-stage differentiated cell, such as a erythrocyte, which plays a characteristic role in a certain tissue type, and may or may not retain the capacity to proliferate further.

The hematopoietic progenitor cell can express at least one of the following cell surface markers characteristic of hematopoietic progenitor cells: CD34⁺, CD59⁺, Thyl/CD90⁺, CD38lo/−, and C-kit/CDl 17⁺. In some examples provided herein, the hematopoietic progenitors can be CD34⁺ cells.

The hematopoietic stem cell can be a peripheral blood stem cell obtained from the patient after the patient has been treated with one or more factors such as granulocyte colony stimulating factor (optionally in combination with Plerixaflor). CD34⁺ cells can be enriched using CliniMACS® Cell Selection System (Miltenyi Biotec). CD34⁺ cells can be stimulated in serum-free medium (e.g., CellGrow SCGM media, CellGenix) with cytokines (e.g., SCF, rhTPO, rhFLT3) before genome editing. Addition of SR1 and dmPGE2 and/or other factors is contemplated to improve long-term engraftment.

Hematopoietic stem cells (HSCs) can be an important target for gene therapy as they provide a prolonged source of the corrected cells. HSCs give rise to both the myeloid and lymphoid lineages of blood cells. Mature blood cells have a finite life-span and must be continuously replaced throughout life. Blood cells are continually produced by the proliferation and differentiation of a population of pluripotent HSCs that can be replenished by self-renewal. Bone marrow (BM) is the major site of hematopoiesis in humans and a good source for hematopoietic stem and progenitor cells (HSPCs). HSPCs can be found in small numbers in the peripheral blood (PB). In some indications or treatments their numbers increase. The progeny of HSCs mature through stages, generating multi-potential and lineage-committed progenitor cells.

In some embodiments, the method comprises delivering a plurality of viral vectors encapsulating one or more nucleic acid sequences and/or polypeptides (e.g., gRNAs targeting a gene of interest such as CCR5 and a nucleic acid encoding a RNA-guided endonuclease) to the stem cells in vivo, thereby editing the target gene of interest in the stem cells. As used herein, the term “viral vector” refer to a virus particle that functions as a nucleic acid delivery vehicle, and which comprises the vector genome packaged within a virion. Exemplary virus vectors of the disclosure include adenovirus vectors, AAVs, lentivirus vectors, retrovirus vectors, and the like.

In some embodiments, the delivering of the one or more nucleic acid sequences occurs after mobilizing the stem cells, such as 0.5 hour, 1 hour, 1.5 hour, 2 hour, 2.5 hour, 3 hours, 4 hours after the administration of the mobilization agents. In some embodiments, the subject is administered with the plurality of viral vectors when a sufficient number of circulating stem cells (e.g., hematopoietic stem cells) may be collected in the blood (e.g., two-fold increase of CD34⁺ cells compared to a control without mobilization). In some embodiments, the delivering of the one or more nucleic acid sequences occurs about 1.5 hour after the administration of the mobilization agents.

Adeno-associated virus (AAV) is a replication-deficient parvovirus, the single-stranded DNA genome of which is about 4.7 kb in length including 145 nucleotide inverted terminal repeat (ITRs). The ITRs play a role in integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

Provided herein include an AAV vector comprising an AAV9 capsid encapsulating (a) one or two gRNAs that targets gene of interest, or a nucleic acid encoding the one or two gRNAs; and (b) a nucleic acid encoding a RNA-guided endonuclease. Also provided herein include a composition comprising a first AAV vector comprising an AAV9 capsid encapsulating one or two gRNAs that target a gene of interest or a nucleic acid encoding the one or two gRNAs; and a second AAV vector comprising an AAV9 capsid encapsulating a nucleic acid encoding a RNA-guided endonuclease. The nucleic acid encoding a RNA-guided endonuclease can be a mRNA of the RNA-guided endonuclease. Disclosed herein includes an AAV vector comprising a nucleic acid encoding (a) one or two gRNAs that target a gene of interest, and (b) a RNA-guided endonuclease. Also disclosed herein include a composition, comprising a first AAV vector comprising a nucleic acid encoding one or two gRNAs that target a gene of interest, and a second AAV vector comprising a nucleic acid encoding a RNA-guided endonuclease. The RNA-guided endonuclease can be, for example, a Cas9 endonuclease, including but not limited to S. pyogenes Cas9 (SpCas9), S. aureus Cas9 (SaCas9), S. lugdunensis Cas9 (SluCas9), N. meningitidis Cas9, S. thermophilus Cas9, S. thermophilus 3 Cas9, T. denticola Cas9, C. jejuni Cas9 (CjCas9), or a variant thereof. The gRNA can be, for example, a single-guide RNA (sgRNA). In some embodiments, the at least one of the one or two gRNAs targets exon 3 of C-C chemokine receptor type 5 (CCR5) gene, for example one or each of the gRNAs can comprise a space sequence of any one of SEQ ID NOs: 3-7 and 14 in Table 3. In some embodiments, the at least one of the one or two gRNAs targets the erythroid specific enhancer of B-cell lymphoma/leukemia 11A (BCL11A) gene. The gene of interest can be, for example, a gene that is preferentially expressed in hematopoietic cells, for example hematopoietic stem cells (HSCs) and/or hematopoietic progenitor cells (HPCs). Non-limiting examples of gene that is preferentially expressed in hematopoietic cells include BCL11A, OCT6 and GATA-2. In some embodiments, the gene of interest is BCL11A.

In some embodiments, the viral vectors can include additional sequences that make the vectors suitable for replication and integration in eukaryotes. In some embodiments, the viral vectors include a shuttle element that makes the vectors suitable for replication and integration in both prokaryotes and eukaryotes. In some embodiments, the viral vectors include additional transcription and translation initiation sequences, such as promoters and enhancers; and additional transcription and translation terminators, such as polyadenylation signals. Various regulatory elements that can be included in an AAV vector have been described in details in US2012/0232133 which is hereby incorporated by reference in its entirety.

In some embodiments, the viral vectors (e.g., AAVs) carrying the one or more nucleotides required for gene editing (e.g., gRNAs targeting CCR5 and the nucleic acid encoding a DNA endonuclease) are administered to the subject at a dose of about 1E14 vector genomes/kilogram (vg/kg) to 5E14/vg/kg per administration. When the viral vectors are administered to the subject two or more times, the dose can be the same or different for each of the administration to the subject.

In some embodiments, a recombinant AAV (rAAV) can be used for delivery. rAAV can be generated by replacing the wildtype AAV open reading frame with a transgene expression cassette. AAVs are small, non-enveloped, single-stranded DNA viruses. The AAV genome is 4.7 kb and is characterized by two inverted terminal repeats (ITR) and two open reading frames which encode the Rep proteins and Cap proteins. The Rep reading frame encodes four proteins, Rep78, Rep68, Rep52, Rep40, which function mainly in regulating the transcription and replication of the AAV genome. The Cap reading frame encodes three structure (capsid) viral proteins (VPs): VP1, VP2 and VP3. The ITRs play a role in integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

Techniques to produce rAAV particles, in which an AAV genome to be packaged including the polynucleotide to be delivered, rep and cap genes, and helper virus functions are provided to a cell (e.g., a packaging cell), are known in the art. Production of rAAV typically requires the following components present within a packaging cell: a transfer plasmid containing the nucleotide(s) to be delivered, a packaging plasmid containing the AAV structural and packaging genes (e.g., rep and cap genes), and a helper plasmid containing the proteins needed for the virus to replicate. The AAV rep and cap genes can be from any AAV serotype for which recombinant virus can be derived, and can be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13 and AAV rh.74. Production of pseudotyped rAAV is known in the art and disclosed in, for example, international patent application publication number WO 01/83692. A packaging cell can then be infected with the three plasmids herein described to produce a rAAV particle encapsulating the one or more nucleic acid (e.g., gRNAs targeting CCR5 and a nucleic acid encoding a DNA endonuclease) to be delivered to the stem cells. A packaging cell or a viral production cell refers to cells used to produce viral vectors. Any viral production cell lines known in the art can be used to produce the viral vectors herein described. HEK293 and 293T cells are common viral production cell lines. Sequences of exemplary AAV serotypes can be obtained from GenBank (see Table 1).

TABLE 1
Exemplary AAV serotype sequences

| AAV serotype | Genbank Accession No. |
|---|---|
| AAV1 | NC_002077.1 |
| AAV2 | NC_001401.2 |
| AAV3 | NC_001729.1 |
| AAV3B | AF028705.1 |
| AAV4 | NC_001829.1 |
| AAV5 | NC_006152.1 |
| AAV6 | AF028704.1 |
| AAV7 | NC_006260.1 |
| AAV8 | NC_006261.1 |
| AAV9 | AX753250.1 |
| AAV10 | AY631965.1 |
| AAV11 | AY631966.1 |
| AAV12 | DQ813647.1 |
| AAV13 | EU285562.1 |

AAV serotypes differ in their tropism, or the types of cells they infect, making AAV a useful system for preferentially transducing specific cell types. Table 2 provides an exemplary summary of the tropism of AAV serotypes, indicating the optimal serotypes for transduction of a given tissue/cell type.

TABLE 2
Exemplary AAV serotypes

| Tissue/Cell type | Serotype |
|---|---|
| Liver | AAV3, AAV5, AAV8, AAV9 |
| Skeletal muscle | AAV1, AAV7, AAV6, AAV8, AAV9 |
| Central nervous system | AAV1, AAV2, AAV4, AAV5, AAV8, AAV9 |
| RPE (Retinal pigment | AAV5, AAV4, AAV2, AAV8, AAV9, |
| epithelium) | AAVrh8R |
| Photoreceptor cells | AAV5, AAV8, AAV9, AAVrh8R |
| Lung | AAV9, AAV5 |
| Heart | AAV8 |
| Pancreas | AAV8 |
| Kidney | AAV2, AAV8 |

In some embodiments, the AAV vector can comprise a polynucleotide to be delivered (e.g., gRNA and/or a nucleic acid encoding Cas9) flanked by a 5′ITR of AAV and a 3′ AAV ITR and a promoter sequence located downstream of the 5′ AAV ITR and upstream of the 3′ AAV ITR. The AAV vector can further comprise one or more polyadenylation signals downstream of the nucleic acid sequence and upstream of the 3′ AAV ITR. The promoter can be, for example, a constitutive promoter or an inducible promoter. A “constitutive” promoter is a promoter that is active under most environmental and developmental conditions. An “inducible” promoter is a promoter that is active under environmental or developmental regulation. In some embodiments, the promoter is a tissue-specific promoter. Exemplary promoters that can be used in the viral vectors described herein include a MND promoter, a U6 promoter, a CMV promoter, a SV40 promoter, a metallothionein promoter, a murine mammary tumor virus (MMTV) promoter, a Rous sarcoma virus (RSV) promoter, a polyhedrin promoter, a chicken 3-actin (CBA) promoter, an EF-1 alpha promoter, a dihydrofolate reductase (DHFR) promoter, a GUSB240 promoter (e.g., a human GUSB240 (hGUSB240) promoter), GUSB379 promoter (e.g., a human GUSB379 (hGUSB379) promoter), and a phosphoglycerol kinase (PGK) promoter (e.g., a human PGK (hPGK) promoter).

In some embodiments, the AAV vector comprises a stuffer sequence. AAV vectors typically accept inserts of DNA having a defined size range which is generally about 4 kb to about 5.2 kb, or slightly more. Thus, for shorter sequences, it may be necessary to include additional nucleic acid in the insert fragment in order to achieve the required length which is acceptable for the AAV vector. The stuffer sequence can be isolated or derived from a non-coding region (e.g., an intronic region) of a known gene or nucleic acid sequence. The stuffer sequence can be for example, a sequence between 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000 nucleotides in length. The stuffer sequence can be located in the nucleic acid or cassette at any desired position such that it does not prevent a function or activity. In some embodiments, the AAV vectors disclosed herein can be used as AAV transfer vectors carrying a transgene encoding a gRNA and/or a DNA endonuclease for producing recombinant AAV viral particles that can be used for delivery.

The AAV serotypes and variants thereof can be used for the nucleic acid delivery include, but not limited to, AAV1, AAV2, AAV3 (including AAV3A and AAV3B), AAV4, AAV5, AAV6, AAV7, AAV8, AAV9, AAV10, AAV11, AAV12, AAV13, AAVrh8R, AAVrh74, AAVrh32.33, AAVrh10, AAVhu.68, avian AAV, bovine AAV, canine AAV, equine AAV, ovine AAV, snake AAV, bearded dragon AAV, AAV2i8, AAV2g9, AAV-LK03, AAV7m8, AAVAnc80, AAVPHP.B, and any other AAV now known or later discovered. See e.g., Bernard N. Fields et al., VIROLOGY, volume 2, chapter 69 (4th ed., Lippincott-Raven Publishers). A number of AAV serotypes and clades have been identified (see e.g., Gao et al, (2004) J. Virology 78:6381-6388; Moris et al, (2004) Virology 33-:375-383).

In some embodiments, AAV9 is used to deliver the one or more nucleic acid herein described to the stem cells. As described herein, AAV9 has tissue tropism for stem cells (e.g., HSCs) and low liver tropism (see e.g., Example 1), and thus are particularly well suited for delivery of the nucleic acids herein described to stem cells.

The one or more nucleic acid herein described (e.g., gRNAs and a nucleic acid encoding a DNA endonuclease) can be encoded in one or more AAV vector. In some embodiments, the gRNA and a nucleic acid encoding a DNA endonuclease (e.g., Cas9) can be encoded in a single AAV vector (see e.g., Example 2). In some embodiments, the gRNA(s) and a nucleic acid encoding a DNA endonuclease (e.g., Cas9) can be encoded into two or more separate AAV vectors. For example, the gRNA(s) can be encoded in one AAV vector and the nucleic acid encoding a DNA endonuclease can be encoded in another AAV vector (see e.g., Example 4). One or more guide RNA can be used with a CRISP/Cas nuclease system. When more than one guide RNA is used, each guide RNA can be encoded on the same or on different vectors.

The gRNA described herein can comprise any suitable spacer sequence complementary to a target sequence in a target gene of interest. In some embodiments, the gRNA can comprise any of the spacer sequences and/or variants thereof described herein in the sections below. For example, the gRNA can comprise a spacer sequence selected from any one of SEQ ID NOs: 3-7 and 14 described herein, and variants thereof having about, at least, at least about 80%, 85%, 90%, 95%, 97%, 98%, 99%, or 100% homology to any spacer of SEQ ID NOs: 3-7 and 14. In some embodiments, the gRNA comprises a spacer sequence selected from any one of SEQ ID NOs: 3-7 and 14, and a variant thereof having no more than 3 mismatches compared to any one of SEQ ID NOs: 3-7 and 14. In some embodiments, the gRNA comprises a spacer sequence of any one of SEQ ID NOs: 3-7 and 14.

In some embodiments, the gRNAs used in the methods herein can comprise two or more gRNAs, each comprising a spacer complementary to a target sequence of the target gene of interest. For example, to edit C-C chemokine receptor type 5 (CCR5) gene the gRNAs used in the methods herein can comprise two or more gRNAs each comprising a spacer sequence selected from any one of SEQ ID NO: 3-7 and 14, variants thereof having at least 85% homology to any one of SEQ ID Nos: 3-7 and 14, and variants having no more than 3 mismatches compared to any one of SEQ ID Nos: 3-7 and 14.

The gRNAs used herein can enhance on-target activity while significantly reducing potential off-target effects (i.e., cleaving genomic DNA at undesired locations other than a target gene). In some embodiments, the off-target binding is reduced by about, at least or at least about 80%, 85%, 90%, 95%, 98%, 99% or 100%.

In some embodiments, the DNA endonuclease is a Cas endonuclease described herein or known in the art. The Cas endonuclease can be naturally-occurring or non-naturally-occurring (e.g., recombinant or with mutations). In some embodiments, the DNA endonuclease is selected from the group consisting of a Casl, CaslB, Cas2, Cas3, Cas4, Cas5, Cas6, Cas7, Cas8, Cas9 (also known as Csnl and Csxl2), CaslOO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, or Cpf1 endonuclease, or a functional derivative thereof. In some embodiments, the DNA endonuclease is a Cas9 endonuclease or a variant thereof. In some embodiments, the Cas9 endonuclease is from Streptococcus pyogenes (SpCas9). In some embodiments, the Cas9 endonuclease is from Staphylococcus lugdunensis (SluCas9). In some embodiments, the Cas9 endonuclease is from Staphylococcus aureus (SaCas9).

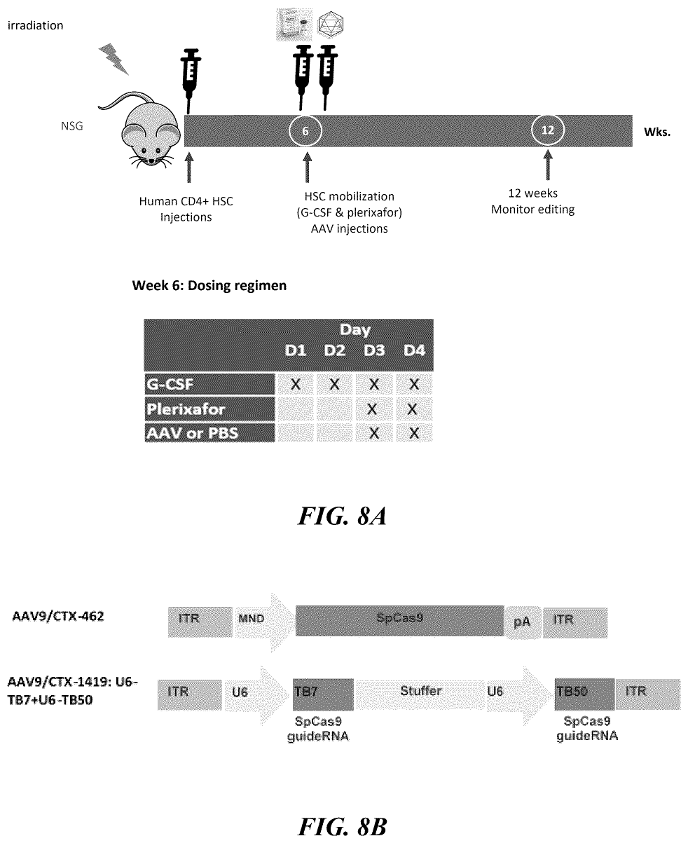

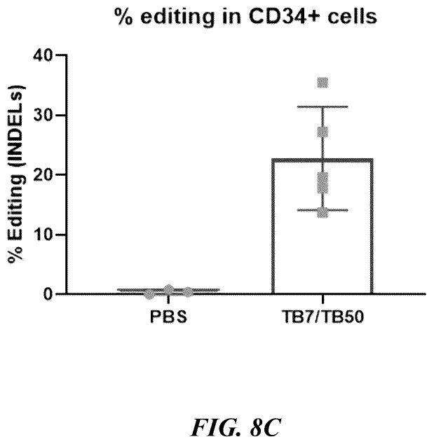

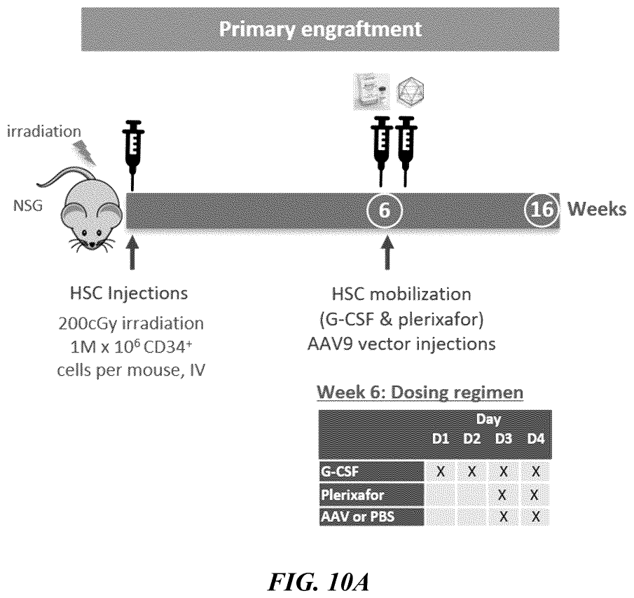

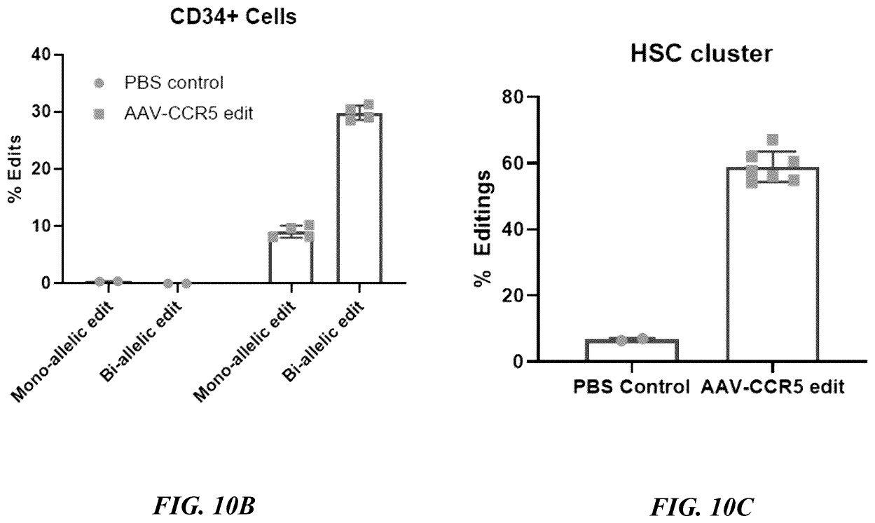

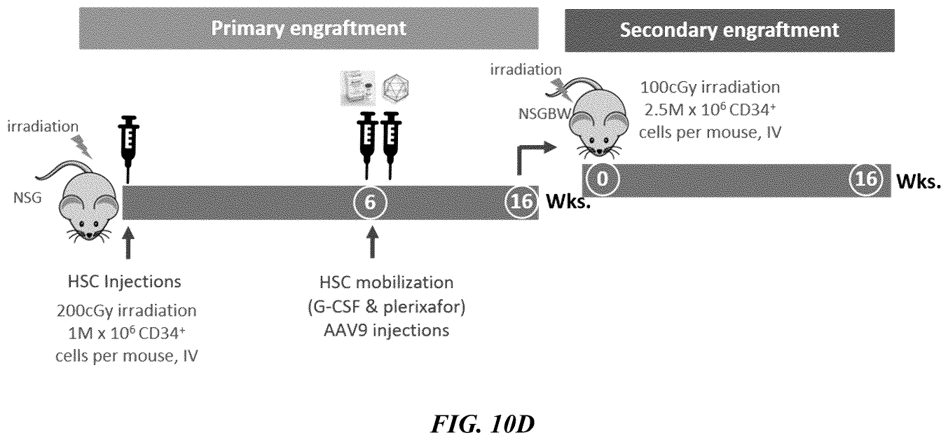

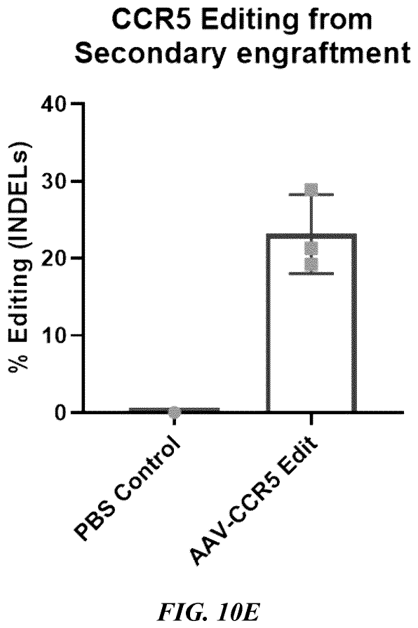

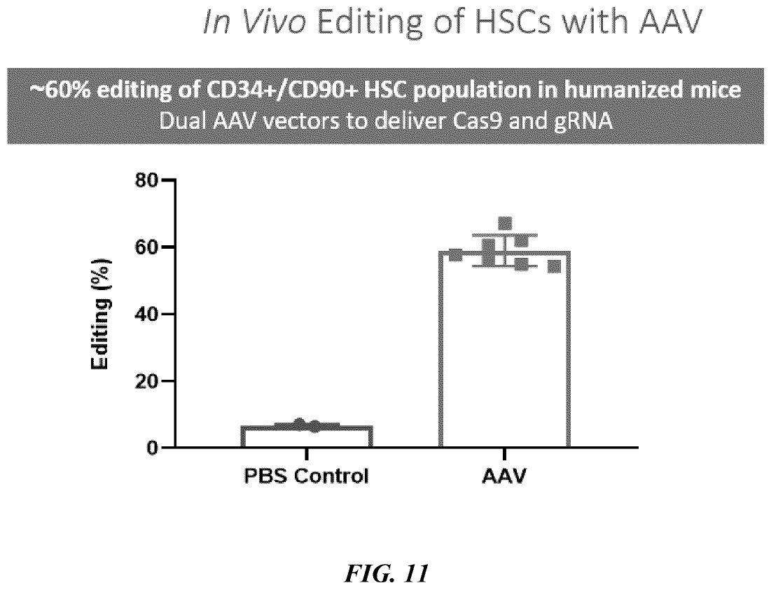

The subject can be administered with the mobilization agents and rAAV particles once, twice, or more times. In some embodiments, the mobilization agents and/or rAAV particles are administered to the subject daily. In some embodiments, the mobilization agents and/or rAAV are administered to the subject in more than one administration cycle (e.g., 3, 4, 5, 6, 7, 8, 9 or 10 administration cycles) with about 1, 2, 3, 4, 5 or 6 days between two administration cycles when no mobilization and/or rAAV particles are administered. In some embodiments, the mobilization agents and/or rAAV are administered to the subject in more than one administration cycle of 1-8 days of daily administration with 2-6 days with no administration of mobilization agents and/or rAAVs. For example, the mobilization agents can be administered to the subject in 6 administration cycles of 4 days of daily administrations with 3 days with no administration of the mobilization agents. In another example, the mobilization agents can be administered to the subject in 6 administration cycles of 2 days of daily administration with 5 days with no administration of the mobilization agents (see e.g., FIG. 8A and FIG. 10A). The rAAVs can be administrated to the subject in 6 administration cycles of 2 daily administration with 5 days with no administration of the rAAVs (see e.g., FIG. 8A and FIG. 10A). The suitable time period between two administrations can be the same as or different from the suitable time period between another two administrations. In some embodiments, the mobilization agents and rAAV particles are administered to the subject daily for two, three, four, five, six, seven, eight or more consecutive days. The mobilization agents and the rAAV particles can be administered to the subject sequentially or concurrently.

In some embodiments, the genetic modification of a target gene (e.g., CCR5 or BCL11A) can result in significantly reduced mRNA and/or protein levels of the target gene in the stem cells of a subject. This reduction can be relative to the gene expression of the subject prior to the gene therapy, a gene expression level in one or more untreated subject, or a reference level of gene expression in a healthy subject. For example, the genetic modification of the target gene can result in a significantly reduced target gene mRNA and/or protein levels. In some embodiments, the target gene expression level is reduced by 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% or a number or a range between any two of these values. This reduction can be relative to a target gene expression of the subject prior to the gene therapy, a target gene expression level in one or more untreated subject, or a reference level of subject having a nonfunctional protein of the target gene (e.g., a CCR5A32 subject).

### Gene Editing

Provided herein includes methods, vectors, compositions and kits for in vivo editing a gene of interest in stem cells, thereby reducing the expression level of the gene (e.g., mRNA or protein expression level) in a subject. Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When an sequence of an endogenous gene is edited, for example by deletion, insertion or substitution of nucleotide(s)/nucleic acid(s), the endogenous gene comprising the affected sequence can be knocked-out or knocked-down due to the sequence alteration. Therefore, targeted editing can be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide can introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Other non-limiting examples of targeted nucleases include naturally-occurring and recombinant nucleases, e.g., CRISPR/Cas9, restriction endonucleases, meganucleases, homing endonucleases, and the like.

### CRISPR-Cas Gene Editing System and RNA-Guided Nuclease

In some embodiments, the methods, and related vectors, compositions and kits described herein can be used in a gene editing system, such as in a CRISPR-Cas gene editing system, to genetically edit a gene of interest (e.g., CCR5 gene or BCL11A gene). For example, the CRISPR-Cas9 system is a naturally-occurring defense mechanism in prokaryotes that has been repurposed as a RNA-guided DNA-targeting platform used for gene editing. It relies on the DNA nuclease Cas9, and two noncoding RNAs-crisprRNA (crRNA) and trans-activating RNA (tracrRNA) to target the cleavage of DNA. crRNA drives sequence recognition and specificity of the CRISPR-Cas9 complex through Watson-Crick base pairing typically with a 20 nucleotide (nt) sequence in the target DNA. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM). TracrRNA hybridizes with the 3′ end of crRNA to form an RNA-duplex structure that is bound by the Cas9 endonuclease to form the catalytically active CRISPR-Cas9 complex, which can then cleave the target DNA. Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR). In some embodiments, CRISPR-Cas9 gene editing system comprises an RNA-guided nuclease and one or more guide RNAs targeting one or more target genes.

As described herein, the RNA-guided endonuclease can be naturally-occurring or non-naturally occurring. The Non-limiting Examples of RNA-guided endonuclease include a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, and functional derivatives thereof. In some instances, the RNA-guided endonuclease is a Cas9 endonuclease. The Cas9 endonuclease can be from, e.g., Streptococcus pyogenes (SpyCas9), Staphylococcus lugdunensis (SluCas9), or Staphylococcus aureus (SaCas9). In some embodiments, the RNA-guided endonuclease is a variant of Cas9, including but not limited to, a small Cas9, a dead Cas9 (dCas9), and a Cas9 nickase. In some embodiments, a Cas nuclease can comprise a RuvC or RuvC-like nuclease domain (e.g., Cpf1) and/or a HNH or HNH-like nuclease domain (e.g., Cas9).

The RNA-guided endonuclease can be a small RNA-guided endonuclease. The small RNA-guided endonucleases can be engineered from portions of RNA-guided endonucleases derived from any of the RNA-guided endonucleases described herein and known in the art. The small RNA-guided endonucleases can be, e.g., small Cas endonucleases. In some cases, a small RNA-guided nuclease is shorter than about 1,100 amino acids in length.

The RNA-guided endonuclease can be a mutant RNA-guided endonuclease. For example, the RNA-guided endonuclease can be a mutant of a naturally occurring RNA-guided endonuclease. The mutant RNA-guided endonuclease can also be a mutant RNA-guided endonuclease with altered activity compared to a naturally occurring RNA-guided endonuclease, such as altered endonuclease activity (e.g., altered or abrogated DNA endonuclease activity without substantially diminished binding affinity to DNA). Such modification can allow for the sequence-specific DNA targeting of the mutant RNA-guided endonuclease for the purpose of transcriptional modulation (e.g., activation or repression); epigenetic modification or chromatin modification by methylation, demethylation, acetylation or deacetylation, or any other modifications of DNA binding and/or DNA-modifying proteins known in the art. In some embodiments, the mutant RNA-guided endonuclease has no DNA endonuclease activity.

The RNA-guided endonuclease can be a nickase that cleaves the complementary strand of the target DNA but has reduced ability to cleave the non-complementary strand of the target DNA, or that cleaves the non-complementary strand of the target DNA but has reduced ability to cleave the complementary strand of the target DNA. In some embodiments, the RNA-guided endonuclease has a reduced ability to cleave both the complementary and the non-complementary strands of the target DNA.

Guide RNAs (gRNAs)

In some embodiments, the CRISPR/Cas-mediated gene editing system used to genetically edit a target gene of interest comprises a genome-targeting nucleic acid (e.g., a guide RNA) that can direct the activities of a RNA-guided endonuclease to a specific target sequence within the target gene of interest. A guide RNA comprises at least a spacer sequence that hybridizes to a target nucleic acid sequence of interest, and a CRISPR repeat sequence. The gRNA can be a single-molecule guide RNA or a double-molecule guide RNA. The RNA-guided endonuclease can be, for example a Cas endonuclease, including Cas9 endonuclease. The Cas9 endonuclease can be, for example, a SpyCas9, a SaCas9, or a SluCas9 endonuclease. In some embodiments, the RNA-endonuclease is a Cas9 variant. In some embodiments, the RNA-guided endonuclease is a small RNA-guided endonuclease. In some embodiments, the RNA-guided endonuclease is a small Cas endonuclease.

In some embodiments, the gRNA comprise 5′ to 3′: a crRNA and a tracrRNA, wherein the crRNA and tracrRNA hybridize to form a duplex. In some embodiments, the crRNA comprises a spacer sequence capable of targeting a target sequence in a target nucleic acid (e.g., genomic DNA molecule) and a crRNA repeat sequence. In some embodiments, the tracrRNA comprises a tracrRNA anti-repeat sequence and a 3′ tracrRNA sequence. In some embodiments, the 3′ end of the crRNA repeat sequence is linked to the 5′ end of the tracrRNA anti-repeat sequence, e.g., by a tetraloop, wherein the crRNA repeat sequence and the tracrRNA anti-repeat sequence hybridize to form the sgRNA. In some embodiments, the sgRNA comprises 5′ to 3′: a spacer sequence, a crRNA repeat sequence, a tetraloop, a tracrRNA anti-repeat sequence, and a 3′ tracrRNA sequence. In some embodiments, the sgRNA comprise a 5′ spacer extension sequence. In some embodiments, the sgRNA comprise a 3′ tracrRNA extension sequence. The 3′ tracrRNA can comprise, or consist of, one or more stem loops, for example one, two, three, or more stem loops.

In some embodiments, the invariable sequence of the sgRNA comprises the nucleotide sequence of GUUUUAGAGCUAGAAAUAGCAAGUUAAAAUAAGGCUAGUCCGUUAUCAACUUGA AAAAGUGGCACCGAGUCGGUGCUUUU (SEQ ID NO: 1), or a nucleotide sequence having up to 1, 2, 3, 4, 5, 6, 7, 8, 9, or 10 nucleotide deletions, insertions, or substitutions relative to SEQ ID NO: 1. In some embodiments, the sgRNA is for use with a SpyCas9 endonuclease.

The guide RNA disclosed herein can target any sequence of interest via the spacer sequence in the crRNA. A spacer sequence in a gRNA is a sequence (e.g., a 20 nucleotide sequence) that defines the target sequence (e.g., a DNA target sequences, such as a genomic target sequence) of a target gene of interest (e.g., CCR5 gene or BCL11A gene). In some embodiments, the spacer sequence range from 15 to 30 nucleotides. For example, the spacer sequence can be, can be about, can be at least, or can be at most 10, 15, 16, 17, 18, 19, 29, 21, 22, 23, 24, 25, 26, 27, 28, 29, 30, 40, 50, or a number or a range between any of these values, of nucleotides in length. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In some embodiments, the gRNA targets within or near a coding sequence in the target gene. In some embodiments, the gRNA targets a sequence within one of the exons of the gene. The gRNA can comprise a spacer sequence complementary to a target sequence within exon 1, 2, or 3 of the target gene. In some embodiments, the gRNA targets within or near a non-coding sequence or a sequence within or near a regulatory element in the target gene. For example, the gRNAs described herein can target a sequence within the erythroid specific enhancer of the BCL11A gene to treat 0-thalassemia and sickle cell disease.

In some embodiments, the spacer(s) are complementary to a sequence within or near (for example, within any of 1, 2, 3, 4, 5, 6, 7, 8, 9, 10, 15, 20, 25, 30, 35, 40, 45, 50, 55, 60, 65, 70, 75, 80, 85, 90, 95, 100 or more bases from) an exon of a target gene. The complementarity between the spacer of the gRNA and the target sequence in the target gene can be perfect or imperfect. In some embodiments, the complementarity can be at least 70%, 80%, 90%, 100% or a number or a range between any two of these values. In some embodiments, the complementarity is perfect, i.e. 100%.

In a CRISPR/Cas system used herein, the spacer sequence is designed to hybridize to a region of the target nucleic acid that is located 5′ of a PAM recognizable by a Cas9 enzyme used in the system. The spacer can perfectly match the target sequence or can have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNNNRG-3′ (SEQ ID NO: 2), the target nucleic acid can be the sequence that corresponds to the Ns, wherein N can be any nucleotide, and the underlined NRG sequence (R is G or A) is the S. pyogenes PAM. In some embodiments, the PAM sequence used in the compositions and methods of the present disclosure as a sequence recognized by SpCas9 is NGG, wherein N can be A, T, C or G.

In some embodiments, the percent complementarity between the spacer sequence and the target nucleic acid is about, at least, at least about, at most or at most about 30%, 40%, 50%, 60%, 65%, 70%, 75%, 80%, 85%, 90%, 95%, 97%, 98%, 99%, or 100%. In some embodiments, the spacer sequence of the guide RNA and the target nucleic acid in the target gene is 100% complementary. In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over about 20 contiguous nucleotides. In some embodiments, the spacer sequence of the guide RNA and the target sequence in the target gene can contain up to 10 mismatches, e.g., up to 9, up to 8, up to 7, up to 6, up to 5, up to 4, up to 3, up to 2, or up to 1 mismatch.

A gene of interest (also referred to as “a target gene” herein) can be any potential therapeutic target for treating or preventing a disease or disorder. In some embodiments, the gene of interest is a C-C chemokine receptor type 5 (CCR5) gene. CCR5 is a transmembrane protein present on the surface of various cells of the immune system, including CD4⁺ helper T cells, macrophages, and dendritic cells. As a chemokine receptor, CCR5, can bind chemokines such as macrophage inflammatory protein-la (MIP-la), MIP-10, and regulated on activation normal T cell expressed and secreted (RANTES). Binding of these chemokine molecules to CCR5 causes signal transduction in the cytosol consistent with CCR5 function as a G protein-coupled receptor. Chemokine receptors are important for directing localization of immune cells to areas of inflammation.

In some embodiments, the gRNA described herein comprises a spacer sequence targeting a sequence in the CCR5 gene. The gRNA described herein can comprise a spacer sequence selected from SEQ ID NOs: 3-7 and 14 listed in Table 3 below or variants thereof having about, at least, at least about 80%, 85%, 90%, 95%, 97%, 98%, 99%, or 100% homology to any spacer of SEQ ID NOs: 3-7 and 14 or variants thereof having no more than 3 mismatches compared to any one of SEQ ID NOs: 3-7 and 14. The spacer sequences set forth in SEQ ID NOs: 3-7 and 14 can target a target sequence set forth in SEQ ID NOs: 8-13, respectively.

TABLE 3
Exemplary spacer/target sequences

| Guide | Sequence | PAM | SEQ ID NO |
|---|---|---|---|
| T13 | AAGCCATGTGCACAACTCTGA (Target) | CTGGGT | 8 (Target) |
| | AAGCCAUGUGCACAACUCUGA (Spacer) | | 3 (Spacer) |
| T10 | CTGACTGGGTCACCAGCCCAC (Target) | TTGAGT | 9 (Target) |
| | CUGACUGGGUCACCAGCCCAC (Spacer) | | 4 (Spacer) |
| TB7 | AGTTTACACCCGATCCACTG (Target) | GGG | 10 (Target) |
| | AGUUUACACCCGAUCCACUG (Spacer) | | 5 (Spacer) |
| TB48 | CAATGTGTCAACTCTTGACA (Target) | GGG | 11 (Target) |
| | CAAUGUGUCAACUCUUGACA (Spacer) | | 6 (Spacer) |
| TB50 | CGACAAAGGCATAGATGATG (Target) | GGG | 12 (Target) |
| | CGACAAAGGCAUAGAUGAUG (Spacer) | | 7 (Spacer) |
| TB8 | TAAACTGAGCTTGCTCGCTC (Target) | GGG | 13 (Target) |
| | TAAACUGAGCUUGCUCGCUC (Spacer) | | 14 (Spacer) |

In some embodiments, the gene of interest is a B-cell lymphoma/leukemia 11A (BCL11A) gene. BCL11A gene encodes a C2H2 type zinc-finger protein by its similarity to the mouse Bcl11a/Evi9 protein. The corresponding mouse gene is a common site of retroviral integration in myeloid leukemia, and may function as a leukemia disease gene, in part, through its interaction with BCL6. During hematopoietic cell differentiation, this gene is down-regulated. It is possibly involved in lymphoma pathogenesis since translocations associated with B-cell malignancies also deregulates its expression. BCL11A has been identified as a key regulator of fetal hemoglobin silencing and a regulator of the developmental fetal-to-adult hemoglobin switch. Increased production of fetal hemoglobin can ameliorate the severity of sickle cell disease and β-thalassemia. Therefore, BCL11A can be a potential therapeutic target for fetal hemoglobin induction to treat sickle cell disease and β-thalassemia.

Accordingly, in some embodiments, the gRNA described herein comprises a spacer sequence targeting a sequence of the BCL11A gene.

In some embodiments, the gRNA comprises a spacer sequence selected from any one of SEQ ID NOs: 3-7 and 14, and variants thereof having about, at least, at least about 80%, 85%, 90%, 95%, 97%, 98%, 99%, or 100% homology to any spacer of SEQ ID NOs: 3-7 and 14. In some embodiments, the gRNA comprises a spacer sequence from any one of SEQ ID NOs: 3-7 and 14 or a variant thereof having no more than 3 mismatches compared to any one of SEQ ID NOs: 3-7 and 14. In some embodiments, the gRNA is a sgRNA.

In some embodiments, more than one guide RNA can be used with a CRISPR/Cas nuclease system. Each guide RNA can contain a different targeting sequence, such that the CRISPR/Cas system cleaves more than one target nucleic acid. In some embodiments, one or more guide RNAs can have the same or differing properties such as activity or stability within the Cas9 RNP complex. Where more than one guide RNA is used, each guide RNA can be encoded on the same or on different vectors.

In some embodiments, two or more gRNAs comprising spacers complementary to a target sequence in the gene of interest are provided to a cell. For example, to edit the CCR5 gene, the gRNAs can comprise any two gRNAs each comprising a spacer selected from the group consisting of SEQ ID NOs: 3-7 and 14 or variants thereof having at least 85% homology to the spacers of SEQ ID NOs: 3-7 and 14 or variants having no more than 3 mismatches compared to any one of SEQ ID NOs: 3-7 and 14. In some embodiments, the gRNAs are any two gRNAs comprising spacers selected from the group consisting of SEQ ID NOs: 5-7 or variants thereof having at least 85% homology to the spacers of SEQ ID NOs: 5-7 or variants having no more than 3 mismatches compared to any one of SEQ ID Nos: 5-7. In some embodiments, the gRNAs comprise a first gRNA comprising a space sequence of SEQ ID NO: 5 or a variant thereof having about, at least, or at least about 80%, 85%, 90%, 95%, 97%, 98%, 99%, or 100% homology to the spacer of SEQ ID NO: 5 and a second gRNA comprising a space sequence of SEQ ID NO: 6 or SEQ ID NO: 7 or a variant thereof having about, at least, or at least about 80%, 85%, 90%, 95%, 97%, 98%, 99%, or 100% homology to the spacer of SEQ ID No: 6 or 7. The two gRNAs can be encoded on the same or on different vectors. In some embodiments, the gRNAs comprise a first gRNA comprising a space sequence of SEQ ID NO: 6 or a variant thereof having about, at least, or at least about 80%, 85%, 90%, 95%, 97%, 98%, 99%, or 100% homology to the spacer of SEQ ID NO: 6 and a second gRNA comprising a space sequence of SEQ ID NO: 7 or a variant thereof having about, at least, or at least about 80%, 85%, 90%, 95%, 97%, 98%, 99%, or 100% homology to the spacer of SEQ ID NO: 7. The two gRNAs can be encoded on the same or on different vectors.

In some embodiments, the gRNA(s) can be encoded on a vector same as or different from the vector encoding the DNA endonuclease. For example, a vector can comprise a nucleic acid encoding a DNA endonuclease and a gRNA or a nucleic acid encoding a gRNA that targets a gene of interest. In some embodiments, two vectors are provided to a subject, one vector comprising a nucleic acid encoding a DNA endonuclease and the other vector comprising one or more gRNA or one or more nucleic acid encoding the one or more gRNA that targets the gene of interest.

In some embodiments, the gRNA is a chemically modified gRNA. Various types of RNA modifications can be introduced to the gRNAs to enhance stability, reduce the likelihood or degree of innate immune response, and/or enhance other attributes as described in the art. The gRNAs described herein can comprise one or more modifications including internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, the chemically-modified gRNA comprises phosphorothioated 2′-O-methyl nucleotides at the 3′ end, the 5′ end, or both of the gRNA. In some embodiments, the chemically-modified gRNA comprises phosphorothioated 2′-O-methyl nucleotides at the 3′ end of the gRNA. In some embodiments, the chemically-modified gRNA comprises phosphorothioated 2′-O-methyl nucleotides at the 5′end of the gRNA. In some embodiments, the chemically-modified gRNA comprises three or four phosphorothioated 2′-O-methyl nucleotides at the 3′ end and/or three or four at the 5′ end of the gRNA. In some embodiments, any one of SEQ ID NOs: 3-7 and 14 can be chemically modified to have three phosphorothioated 2′-O-methyl nucleotides at the 3′ end and three at the 5′ end of the gRNA.

In some embodiments, the gRNAs described herein can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Polynucleotides constructs and vectors can be used to in vitro transcribe a gRNA described herein.

### Pharmaceutical Compositions and Therapeutic Applications

Provided herein also includes a pharmaceutical composition for carrying out the methods disclosed herein. A pharmaceutical composition can comprise the recombinant viral particles (e.g., AAV particles) described herein encapsulating one or more gRNA(s), a RNA-guided endonuclease or a nucleotide sequence encoding the RNA-guided endonuclease described herein. In some embodiments, the viral particles can further comprise a polynucleotide to be inserted (e.g., a donor template) in the gene of interest (e.g., CCR5 or BCL11A) to affect the desired genetic modification of the methods disclosed herein.

The one or more gRNA can comprise any suitable spacer sequence complementary to a target sequence in a target gene of interest. In some embodiments, the one or more gRNA(s) each comprises a spacer complementary to a genomic sequence within or near (for example, within any of 1, 2, 3, 4, 5, 6, 7, 8, 9, 10, 15, 20, 25, 30, 35, 40, 45, 50, 55, 60, 65, 70, 75, 80, 85, 90, 95, 100 or more bases from) a coding or non-coding region of the target gene of interest. In some embodiments, the gRNA can comprise any of the spacer sequences and/or variants thereof described herein. In some embodiments, a gRNA comprises a space sequence of any one of SEQ ID NOs: 3-7 and 14, or a variant thereof having at least 85% homology to the spacer sequence of any one of SEQ ID NOs: 3-7 and 14.

In some embodiments, the RNA-guided endonuclease is selected from the group consisting of a Casl, CaslB, Cas2, Cas3, Cas4, Cas5, Cas6, Cas7, Cas8, Cas9 (also known as Csnl and Csxl2), CaslOO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, and Cpf1 endonuclease, or a functional derivative thereof. In some embodiments, the DNA endonuclease is Cas9. In some embodiments, the Cas9 endonuclease is SpCas9. In some embodiments, the Cas9 endonuclease is from SluCas9. In some embodiments, the Cas9 endonuclease is from SaCas9. In some embodiments, a DNA sequence that is transcribed to the nucleic acid encoding the DNA endonuclease is codon optimized. In some embodiments, the nucleic acid encoding the DNA endonuclease comprises a 5′ CAP structure and 3′ polyA tail.

A composition described above can further have one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

In some embodiments, a composition is formulated with pharmaceutically acceptable excipients such as carriers, solvents, stabilizers, adjuvants, diluents, etc., depending upon the particular mode of administration and dosage form.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by standard clinical techniques.

In some embodiments, the composition herein described can be administered to a subject in need thereof to treat a disease or disorder by creating permanent changes to the genome of a human stem cell in the subject. The vectors, compositions and methods herein described can result in a deletion, insertion, modulation or inactivation of a target gene of interest or a regulatory element of the target gene, which can eliminate or decrease the expression level of the target gene, thereby preventing or treating a disease or disorder.

In some embodiments, the vectors, composition and methods herein described can functionally knock out a target gene (e.g., CCR5 gene or BCL11A gene) in the genome of a relevant cell type in patients (e.g., stem cells) by permanently deleting or editing a target sequence from the target gene or inserting an exogenous sequence in the target gene.

Disclosed herein includes a method comprising administering to a subject in need an effective amount of a mobilization agent and administering to the subject a plurality of viral vectors (e.g., AAVs) encapsulating a guide RNA (gRNA) or a nucleic acid encoding a gRNA that targets CCR5 gene and a nucleic acid encoding a RNA-guided endonuclease. The mobilization agent is provided in an amount effective to mobilize the stem cells into the peripheral blood of the subject in need such that the mobilized stem cells (e.g., hematopoietic stem cells) can be transduced with the viral vectors carrying the genome-targeting gRNA and the nucleic acid encoding the RNA-guided endonuclease. Administering the mobilization agent and the viral particles can be performed sequentially or concurrently. In some embodiments, the viral particles are administered to the subject following the administration of the mobilization agent (e.g., 1 hour, 1.5 hour, 2 hours, 3 hours, 4 hours, or 5 hours after the administration of the mobilization agent) such that a sufficient number of hematopoietic stem cells (e.g., CD34⁺ cells) circulates in the blood (e.g., 2.0×10⁶ CD34⁺ cells/kg or more). In some embodiments, the viral particle is AAV such as AAV9.

In some embodiments, a gRNA or a nucleic acid encoding the gRNA that targets CCR5 gene comprises a space sequence of any one of SEQ ID NOs: 3-7 and 14 or a variant thereof having at least 85% homology to the spacer sequence of any one of SEQ ID NOs: 3-7 and 14. In some embodiments, two or more gRNAs are provided to a subject, each comprising a spacer sequence of any one of SEQ ID NOs: 3-7 and 14 or a variant thereof having at least 85% homology to the spacer sequence of any one of SEQ ID NOs: 3-7 and 14. In some embodiments, the gRNAs comprise a first gRNA comprising a spacer of SEQ ID NO: 5 or a variant thereof having at least 85% homology to the spacer having a sequence of SEQ ID NO: 5 and a second gRNA comprising a space or SEQ ID NO: 6 or 7, or a variant thereof having at least 85% homology to the spacer having a sequence of SEQ ID NO: 6 or 7. In some embodiments, the gRNAs comprise a first gRNA comprising a spacer of SEQ ID NO: 6 or a variant thereof having at least 85% homology to the spacer having a sequence of SEQ ID NO: 6 and a second gRNA comprising a space or SEQ ID NO: 7 or a variant thereof having at least 85% homology to the spacer having a sequence of SEQ ID NO: 7.

The subject can refer to any individual for whom diagnosis, treatment or therapy is desired. The subject can be an individual diagnosis to be at a risk of developing or suffer from a disease or disorder that can be treated by gene editing of the gene of interest. In some embodiments, the subject is a mammal. In some embodiments, the subject is a human being.

The methods and compositions herein described can be administered to a subject with a disease or disorder that can be alleviated or treated by editing one or more genomic regions of interest, for example genomic regions of interest comprising one or more genes of interest (i.e., target genes). Non-limiting examples of the disease or disorder include achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, Crigler-Najjer Syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), Glycogen Storage Disease Type IV, hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, Huntington's disease, X-linked gammaglobulinemia, X-linked hyper IgM syndrome, and X-linked lymphoproliferative syndrome. In some embodiments, the disease or disorder is acquired immunodeficiencies, lysosomal storage diseases (e.g., Gaucher's disease, GM1, Fabry disease and Tay-Sachs disease), mucopolysaccahidosis (e.g., Hunter's disease, Hurler's disease), hemoglobinopathies (e.g., sickle cell diseases, HbC-α-thalassemia, β-thalassemia) and hemophilias. In some embodiments, the disease or disorder is sickle cell disease. The target gene can be, for example, BCL11A or 3-globin gene, for treating sickle cell disease. The target genomic region of interest can be a Huntington's locus, e.g., an HTT gene (e.g., a HTT gene having a CAG repeat expansion comprising more than 35 CAG repeats), for treating Huntington's disease.

The pharmaceutical composition can be administered by, for example, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, and/or intradermal injection, or any combination thereof. The administration can be local or systemic. The systemic administration includes enteral and parenteral administration. In some embodiments, more than one administration can be employed to achieve the desired level of gene expression over a period of various intervals, e.g., daily, weekly, monthly, or yearly.

The pharmaceutical composition thereof can be administered to a subject in need thereof at a pharmaceutically effective amount. The term “pharmaceutically effective amount” as used herein means that the amount of the pharmaceutical composition that will elicit a desired therapeutic effect and/or biological or medical responses of a tissue, system, animal or human. The administration can result in a desired reduction in the expression of the target gene such as a desired reduction in the levels of the target protein. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The compositions and methods herein described can lead to improve or ameliorate one or more signs associated with a disease or disorder that can be improved or ameliorated by editing one or more genomic regions of interest, alleviate one or more symptoms of a disease or disorder that can be alleviated by editing one or more genomic regions of interest, treat a disease or disorder that can be treated by editing one or more genomic regions of interest, and/or delay or prevent the development of a disease or disorder that can be treated by editing one or more genomic regions of interest. In some embodiments, the compositions and methods can delay the progression of the disease, increasing the quality of life and/or prolonging survival (e.g., by 6 months, 1 year, 2 years, 5 years, 10 years, 15 years, 20 years or longer).

In some embodiments, the expression level of the target gene (e.g., mRNA and/or protein level) in the hematopoietic stem cells of the subject following carrying out the method is reduced by about, at least or at least about 10%, 15%, 20%, 25%, 30%, 35%, 40%, 45%, 50%, 55%, 60%, 65%, 70%, 75%, 80%, 85%, 90%, 95%, 98%, or a number or a range between any of these values. In some embodiments, the composition and methods can decrease the amount of functional target gene and/or functional protein product of the target gene in an individual (e.g., by about, at least, or at least about 10%, 15%, 20%, 25%, 30%, 35%, 40%, 45%, 50%, 55%, 60%, 65%, 70%, 75%, 80%, 85%, 90%, 95%, 98%, or a number or a range between any of these values).

In some embodiments, the methods and compositions herein described can be used to treat a subject with a blood disorder such as sickle cell disease. Sickle cell disease refers to a group of inherited red blood cell disorders when a child receives two sickle cell genes, one from each parent. With sickle cell disease, red blood cells contort into a sickle shape. The cells die early, leaving a shortage of healthy red blood cells, and can block blood flow causing pain. Symptoms of sickle cell disease include infections, pain, and fatigue. Sickle cell disease can be diagnosed with a blood test. Common types of sickle cell disease include HbSS also referred to as sickle cell anemia, HbSC, HbS beta thalassemia, HbSD, HbSE and HbSO. Patients with HbSS inherit two sickle cell genes (“S”), one from each parent. Patients with HbSC inherit a sickle cell gene (“S”) from one parent and from the other parent a gene for an abnormal hemoglobin called “C”. Patients with HbS beta thalassemia inherit one sickle cell gene (“S”) from one patent and one gene of beta thalassemia from the other parent. There are two types of beta thalassemia: “0” and “+”. Those with HbS beta 0-thalassemia usually have a severe form of sickle cell disease. Those with HbS beta+-thalassemia tend to have a milder form of sickle cell disease. Patients with HbSD, HbSE or HbSO inherit one sickle cell gene (“S”) and one gene from an abnormal type of hemoglobin (“D”, “E”, or “O”). In some embodiments, the subject in need is a subject having sickle cell trait. A subject having sickle cell trait inherit one sickle cell gene (“S”) from one parent and one normal gene from the other parent. Subjects with sickle cell trait typically do not have any of the signs of the disease, but can pass the trait on to their children.

A method of treating sickle cell disease in a subject in need thereof is also disclosed. The method can comprise administering to a subject in need an effective amount of a mobilization agent and administering to the subject a plurality of viral vectors (e.g., AAVs) encapsulating a guide RNA (gRNA) or a nucleic acid encoding a gRNA that targets BCL11A gene and a nucleic acid encoding a RNA-guided endonuclease, thereby treating the sickle cell disease in the subject. The mobilization agent is provided in an amount effective to mobilize the stem cells into the peripheral blood of the subject in need such that the mobilized stem cells (e.g., hematopoietic stem cells) can be transduced with the viral vectors carrying the genome-targeting gRNA and the nucleic acid encoding the RNA-guided endonuclease. Administering the mobilization agent and the viral particles can be performed sequentially or concurrently. In some embodiments, the viral particles are administered to the subject following the administration of the mobilization agent (e.g., 1 hour, 1.5 hour, 2 hours, 3 hours, 4 hours, or 5 hours after the administration of the mobilization agent) such that a sufficient number of hematopoietic stem cells (e.g., CD34⁺ cells) circulates in the blood (e.g., 2.0×10⁶ CD34⁺ cells/kg or more). In some embodiments, the viral particle is AAV such as AAV9.

Provided herein also includes kits for carrying out the methods described herein. A kit can include one or more mobilization agents (e.g., plerixafor and/or G-CSF) and a viral particle (e.g., AAV) encapsulating a genome-targeting nucleic acid and a nucleic acid encoding a RNA-guided endonuclease. In any of the above kits, the kit can further comprise a polynucleotide to be inserted to effect the desired genetic modification (e.g., a donor template). Components of a kit can be in separate containers, or combined in a single container.

Any kit described above can further comprise one or more additional reagents selected from a buffer, a buffer for introducing the viral particle into a cell, a transfection reagent, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the endonuclease, or improve the specificity of targeting.

In some embodiments, a kit can further include instructions for using the components of the kit to practice the methods described herein. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g., CD-ROM, diskette, flash drive, etc. In some instances, the actual instructions are not present in the kit, but means for obtaining the instructions from a remote source (e.g., via the Internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

Some aspects of the embodiments discussed above are disclosed in further detail in the following examples, which are not in any way intended to limit the scope of the present disclosure.

### Example 1

### Identification of Hematopoietic Stem Cell (HSC) Tropic AAV Capsid

This example screens and selects AAV vectors that show a strong tropism towards HSCs in vivo.

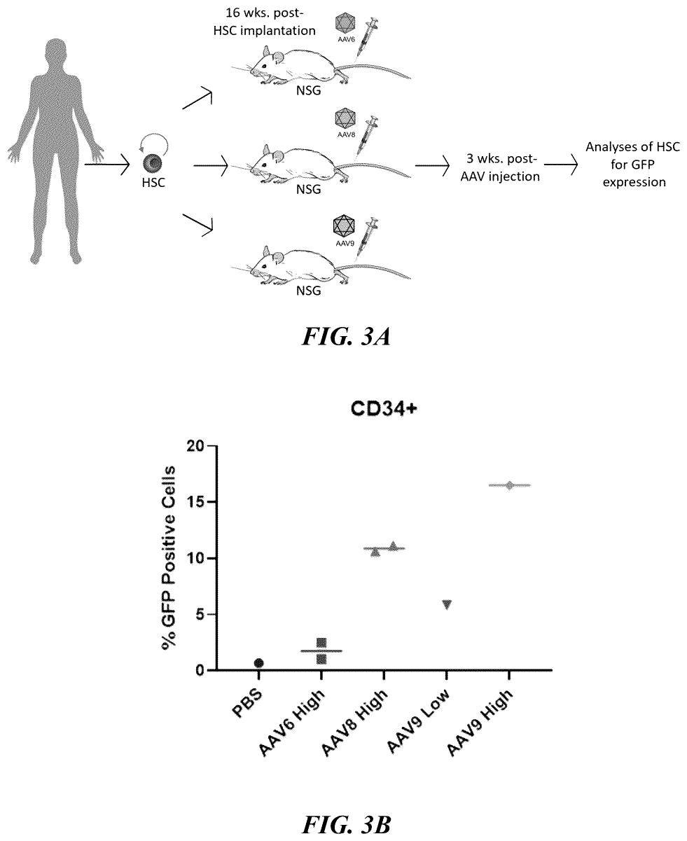

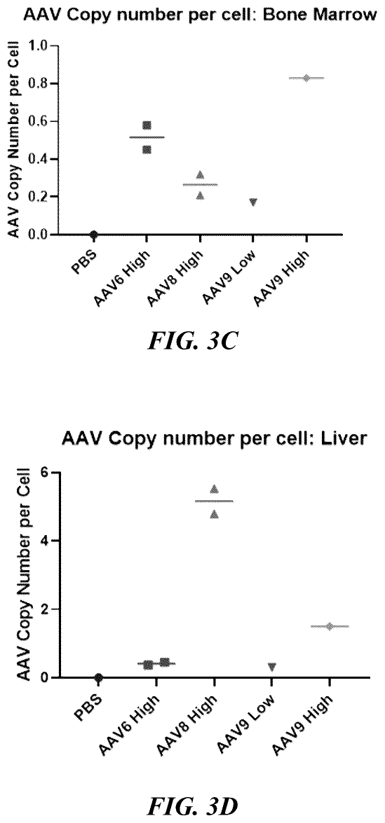

AAV vector plasmid encoding GFP was packaged into AAV6, AAV8, and AAV9 capsids. NSG mice were implanted with human HSCs. Sixteen weeks after implantation, the vectors were intravenously injected into HSC implanted NSG mice (see FIG. 3A). HSCs were analyzed for GFP expression 3 weeks post-AAV injection.

The GFP expression data revealed that AAV8 and AAV9 vectors can transduce more than 10% of CD34⁺ HSPCs (FIG. 3B). The AAV copy number analysis further revealed lower liver tropism with AAV9 than AAV8 in mice (FIG. 3D). AAV9 was identified as optimal capsid for delivery of gene editing components to human HSCs.

### Example 2

### Screening SaCas9 gRNAs for CCR5 Disruption in HSCs

This example screens and selects SaCas9 gRNAs for disruption of CCR5 ORF in HSCs.

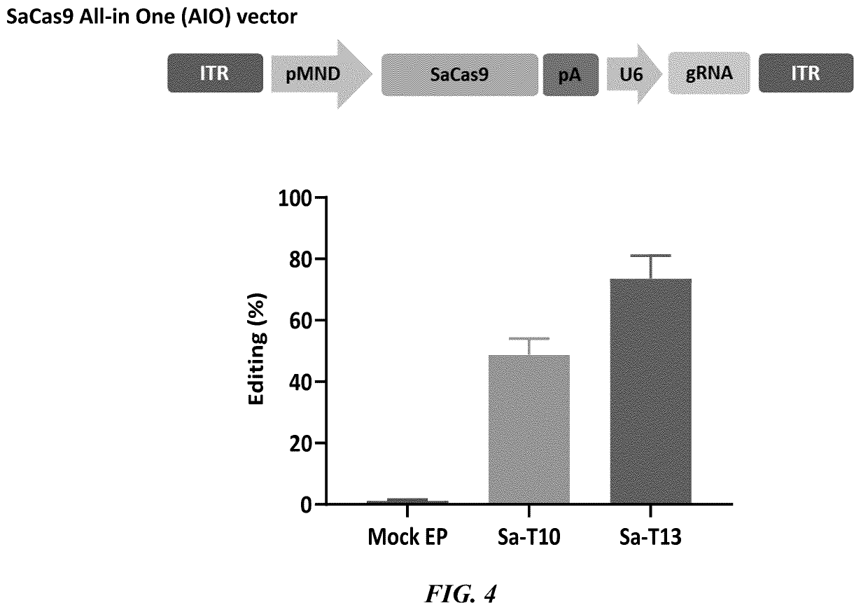

A number of SaCas9 gRNAs targeting exon 3 of the CCR5 gene was screened for editing efficiency in HSCs. gRNAs with high editing efficiency were used to construct a CRISPR/Cas9 system. Human CD34⁺ HSCs were electroporated with Cas9 protein and guide RNA. Two days post-electroporation, genomic DNA was isolated, and editing was analyzed using TIDE PCR. The compact size of the SaCas9 allows the use of the all-in-one AAV vector for in vivo gene editing. The construct of the all-in-one vector used in this example is illustrated in FIG. 4 (middle panel), which comprises a nucleic acid encoding SaCas9 and gRNA targeting exon 3 of the CCR5 gene. Two gRNAs, T10 (48.8% editing efficiency) and T13 (73.63% editing efficiency), with >40% editing efficiency were selected for in vivo gene editing.

### Example 3

### Identification of Optimal HSC Mobilization and AAV Vector Dosing Regimen

Experiments were carried out in this example to identify the optimal HSC mobilization and AAV vector dosing regimen.

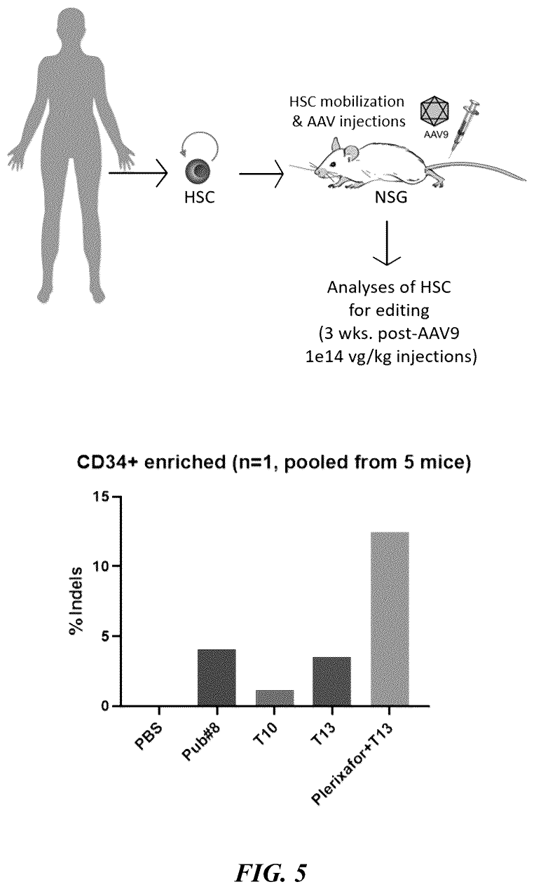

NSG mice were implanted with human HSCs. Four to six weeks after implantation, AAV9 vector(s) expressing SaCas9 and gRNA (e.g., the all-in-one AAV9 vector described in Example 2) was administered intravenously at 1e14 vg/kg body weight into the mice with or without mobilization of HSCs. Different HSC mobilization regimen were examined: plerixafor alone, G-CSF alone, or a combination of G-CSF and plerixafor. Plerixafor and/or G-CSF was administered subcutaneously to the mice at 5 mg/kg (about 100 μg/mouse). In one embodiment (FIG. 5), CD34⁺ enriched cells were isolated from the bone marrows to monitor persistence of CCR5 gene editing in CD34⁺ HSPCs. CCR5 gene editing efficiency was measured by determining the INDEL frequency using TIDES analysis.

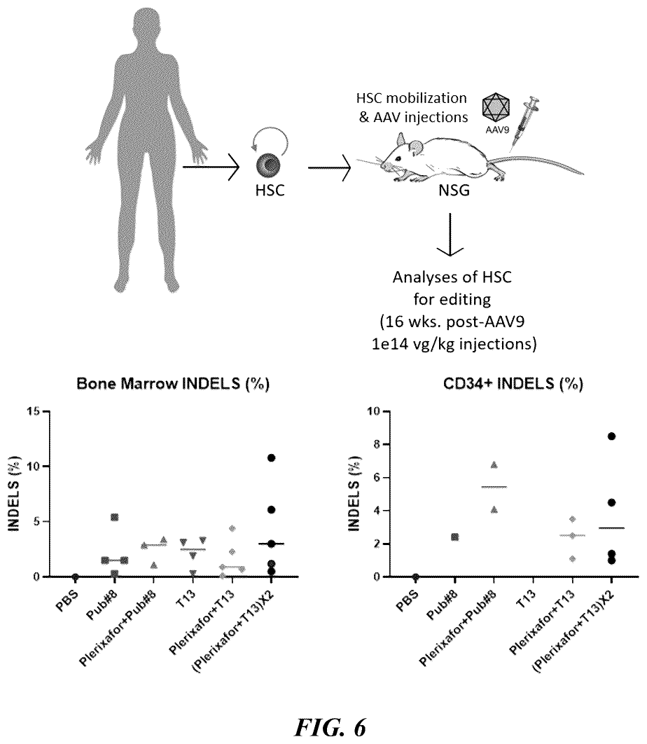

FIG. 6 illustrates a sixteen-week study in which the amount of the plerixafor and AAV vector are doubled. Sixteen weeks after vector administration, CCR5 gene editing were quantified in bone marrow and CD34⁺ HSPCs of the mice.

The results demonstrate that a higher CCR5 editing rate (about 12.5%) is noted in plerixafor mobilized CD34⁺ HSPC group in comparison to groups without mobilization (e.g., plerixafor+T13 vs. T10 or T13 in FIG. 5). The results also demonstrate that increasing the amount of plerixafor and AAV vector results in higher CCR5 editing rate (e.g., up to 10.5% in bone marrow) (FIG. 6). In CD34⁺ HSPCs, the CCR5 editing rate increases from 2.36% to 3.85% when the amount of plerixafor and AAV vector is doubled.

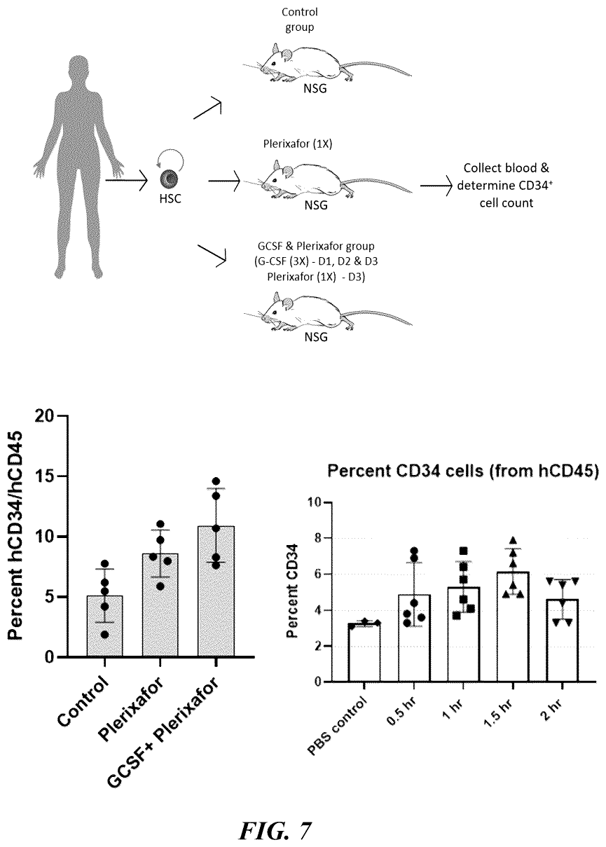

In another embodiment, NGS mice implanted with human HSCs were divided into three groups: the control group was not administered with any mobilization agent; one group was administered with plerixafor one time (1×); and another group was administered with G-CSF three times (3×) at day 1, 2 and 3 and with plerixafor one time at day 3 (FIG. 7). Blood was collected and human CD34⁺ progenitor cells and CD45⁺ cells were counted.

The results demonstrate superiority of G-CSF and plerixafor combination for mobilization of HSCs in humanized NSG mice (FIG. 7, bottom left). The data also shows that plerixafor and G-CSF mediated HSC mobilization was maximal at 1.5 hours post-plerixafor injection (FIG. 7, bottom right).

### Example 4

### Evaluation of an all-In-Two SpCas9 Vector for CCR5 Disruption in HSCs

This example evaluates the CCR5 editing efficiency of an all-in-two SpCas9 vector in HSCs.

Irradiated NSG mice were implanted with human HSCs. At week 6, a first AAV9 vector expressing SpCas9, a second AAV9 vector expressing two gRNAs (TB7 and TB50), G-CSF, and plerixafor were administered into the mice according to the dosing regimen shown in FIG. 8A. In particular, G-CSF was administered at day 1, day 2, day 3 and day 4, and plerixafor and AAV vectors were administered at day 3 and day 4. The constructs of the two AAV vectors used in this example are illustrated in FIG. 8B. Six weeks after the administration (i.e. at week 12), CCR5 gene editing efficiency was measured in CD34⁺ cells of the mice by determining the INDEL frequency using TIDES analysis (FIG. 8C).

The results demonstrate that compared to the all-in-one SaCas9 vector, the all-in-two SpCas9 vector described herein resulted in higher editing efficiency up to 35.5%.

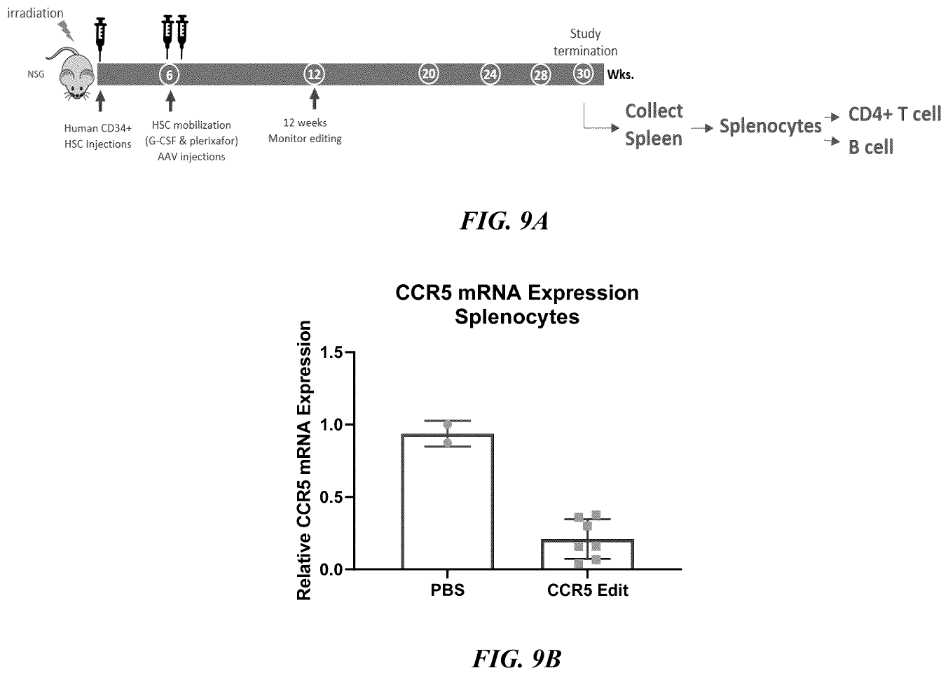

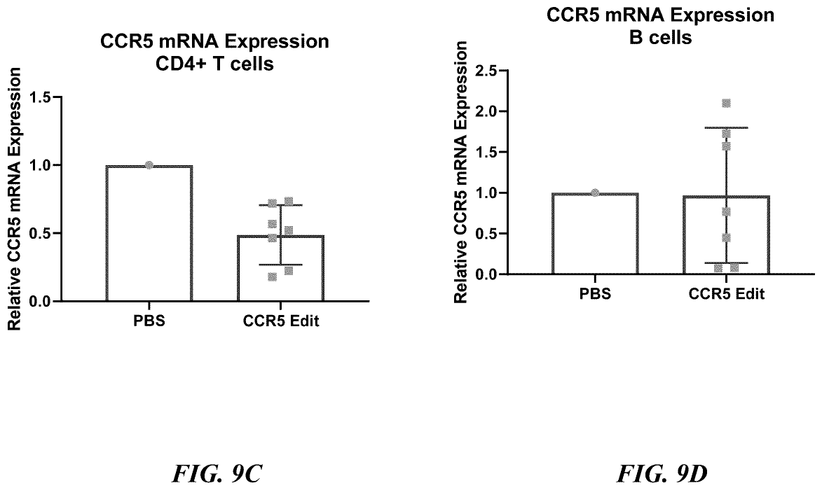

At week 31, the spleen of the mice was collected, splenocytes, CD4⁺ T cells and B cells were isolated from the spleen, and CCR5 mRNA expression level was measured (FIG. 9A).

FIGS. 9B-D show relative CCR5 mRNA expression level in splenocytes (79% reduction of CCR5 mRNA expression; FIG. 9B), CD4⁺ T cells (51% reduction of CCR5 mRNA expression; FIG. 9C) and B cells (FIG. 9D) of the humanized mice treated with AAV9/CTX-1419 and a PBS control. The results demonstrate that the CCR5 mRNA expression decreased in CD4⁺ T cells and splenocytes isolated from spleens of humanized and edited NSG mice.

### Example 5

### Evaluation of CCR5 Editing Efficiency in Long-Term HSCs

This example evaluates the CCR5 editing efficiency and persistency of an non-limiting exemplary all-in-two SpCas9 vector in true long-term HSCs.

NSG mice were irradiated (e.g., 200 cGy total body irradiation) and implanted with human HSCs (e.g., at a dose of 1M×10⁶ CD34⁺ cells per mouse via intravenous injection). At week 6, a first AAV9 vector expressing SpCas9, a second AAV9 vector expressing two gRNAs (TB7 and TB50), G-CSF, and plerixafor were administered into the mice according to the dosing regimen shown in FIG. 10A. In particular, G-CSF was administered at day 1, day 2, day 3 and day 4 each week during the 6-week administration period, and plerixafor and AAV vectors were administered at day 3 and day 4 each week. The constructs of the two AAV vectors used in this example are illustrated in FIG. 8B. Ten weeks after the administration of the vector and mobilization agents (i.e. at week 16), CCR5 gene editing efficiency was measured in CD34+ cells of the mice using single cell DNA sequencing (FIG. 10B) and HSC cluster analysis (FIG. 10C).

Single cell DNA sequencing shows about 40% editing in total CD34⁺ population: 30% bi-allelic and 10% mono-allelic editing (FIG. 10B). Cluster analysis shows 58.96% editing in CD34⁺/CD90⁺ HSC cluster, including indels and deletions (FIG. 10C).

A secondary engraftment was carried out on the mice at week 16. In particular, the NSG mice was irradiated with 100 cGy irradiation and injected with 2.5 M×10⁶ CD34⁺ cells per mouse via intravenous injection. 16 weeks following the secondary engraftment, CCR5 gene editing efficiency was measured in CD34⁺ cells of the mice by determining the INDEL frequency (FIG. 10E).

The INDEL frequency data indicates that secondary engraftment data results in an average 23.1% editing efficiency in long-term HSCs, ranging between 19.2% and 28.9% (FIG. 10E).

Taken together, the results shown in this example demonstrate that the in vivo HSC editing described herein can achieve CCR5 editing efficiency and persistency in long-term HSCs.

### Example 6

### Identification of Optimal CCR5-Specific gRNAs Through in Silico Discovery Pipeline

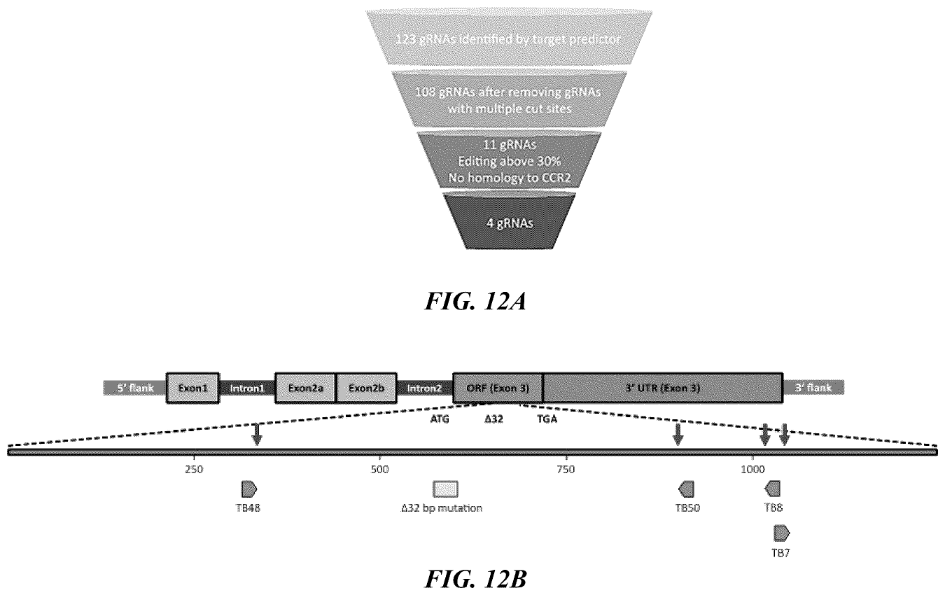

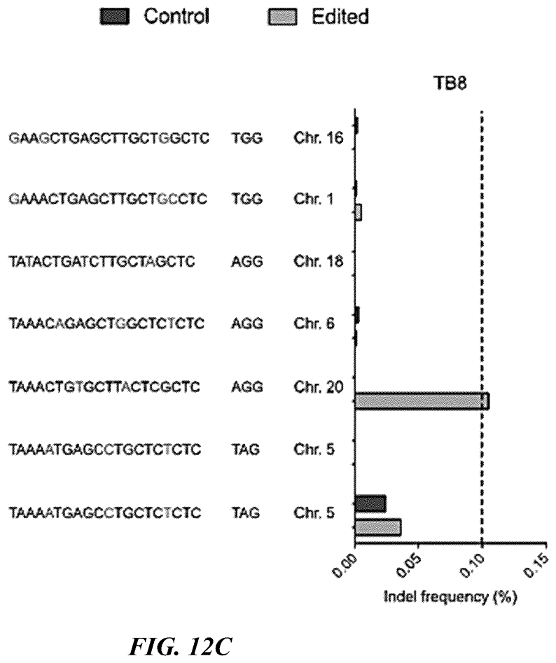

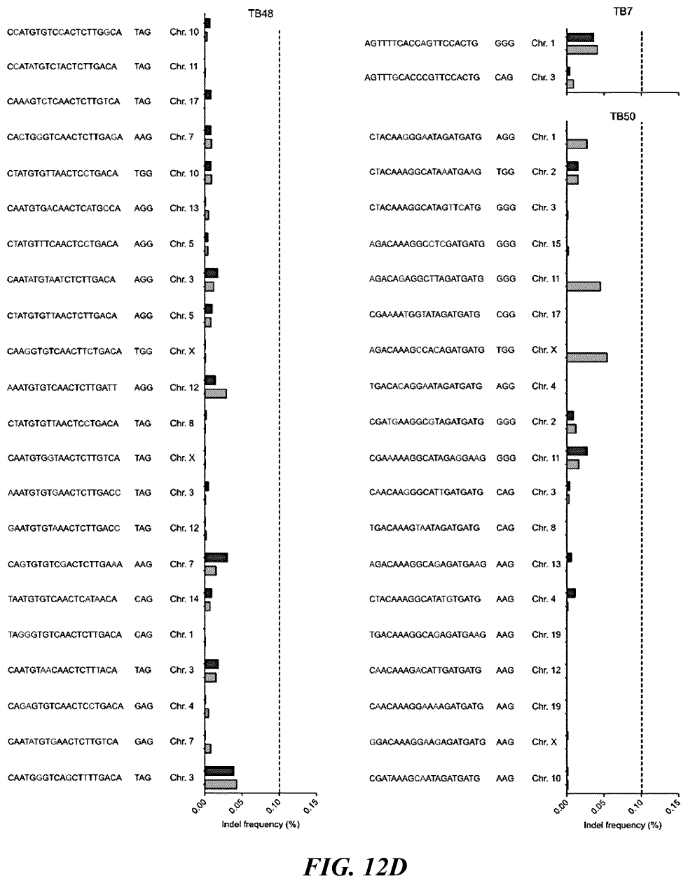

In this example, a combination of in silico prediction software and in vitro screening methods were used to develop guide RNAs (gRNAs) capable of efficiently editing the human ccr5 gene (FIG. 12A).

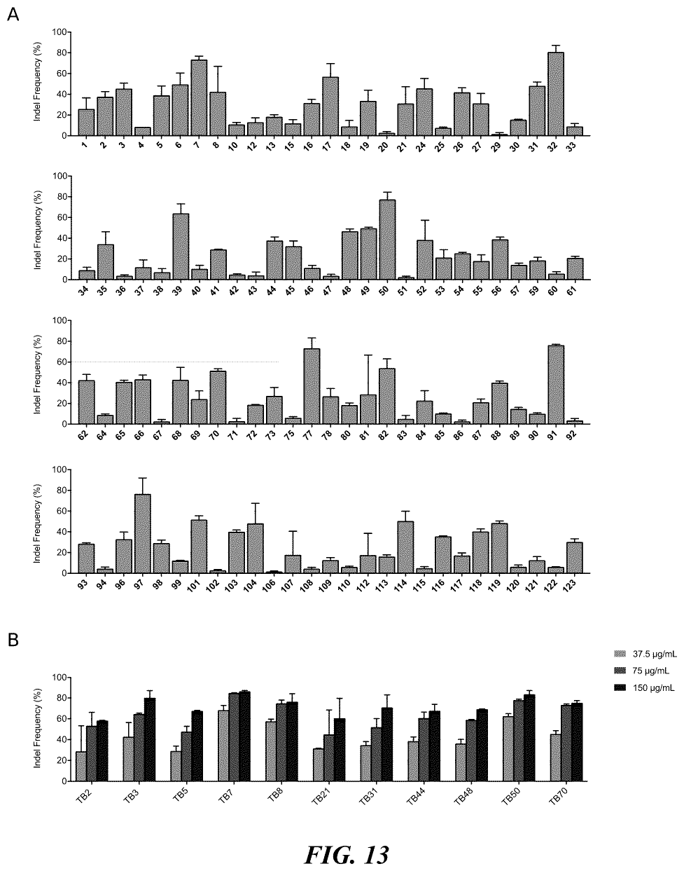

In silico target prediction identified 123 gRNAs with the ability to cause double stranded DNA breaks in the open reading frame (exon 3) of the ccr5 gene. In silico off-target site prediction excluded 15 gRNAs with multiple binding sites in the human genome. The remaining 108 gRNAs were transcribed in vitro and evaluated for editing efficiency when complexed with SpCas9 protein (Cas9) in human CD34⁺ hematopoietic stem cells (HSCs) (FIG. 13, panel A). The 11 gRNAs displaying the highest editing efficiency (editing efficiency >30%) were further evaluated in an optimized chemically synthesized format at increasing dosages of gRNA (FIG. 13, panel B).

The four gRNAs displaying the highest editing efficiency and without apparent sequence homology to other human genes were selected for more stringent off-target editing evaluation (FIG. 12B). Table 4 below provides the gene names and off-targets tested for the four gRNAs TB7, TB8, TB48, or TB50. To evaluate potential off-target editing, CD34⁺ HSCs were either mock edited with Cas9 electroporation only, or electroporated with Cas9 complexed to either TB7, TB8, TB48, or TB50 gRNAs. Off-target gene regions containing sites with <4 base pair mismatches were amplified and deep sequenced to analyze indel frequency formation.

TABLE 4
Off-targets evaluated for exemplary gRNAs.
TB50 Off-Targets

| | | | | | SEQ |
|---|---|---|---|---|---|
| | | | | | ID |
| Chromosome | Gene Name | start | end | Sequence | NO |
| chr1 | LMNA | 156098146 | 156098208 | CTACAAGGGAATAGATGATG | 23 |
| chr2 | RP11- | 152533920 | 152533982 | CTACAAAGGCATAAATGAAG | 24 |
| | 17G11.1 | | | | |
| chr3 | P3H2 | 189956449 | 189956511 | CTACAAAGGCATAGTTCATG | 25 |
| chr15 | RP11-62C7.2 | 39035219 | 39035281 | AGACAAAGGCCTCGATGATG | 26 |
| chr11 | GRM5-AS1 | 88487883 | 88487945 | AGACAGAGGCTTAGATGATG | 27 |
| chr17 | KPNB1 | 47673627 | 47673689 | CGAAAATGGTATAGATGATG | 28 |
| chrX | FAM104B | 55155109 | 55155171 | AGACAAAGCCACAGATGATG | 29 |
| chr4 | RP11- | 133926916 | 133926978 | TGACACAGGAATAGATGATG | 30 |
| | 427M20.1 | | | | |
| chr2 | CXCR1 | 218164267 | 218164329 | CGATGAAGGCGTAGATGATG | 31 |
| chr11 | APLNR | 57236048 | 57236110 | CGAAAAAGGCATAGAGGAAG | 32 |
| chr3 | ADAMTS9- | 64794052 | 64794114 | CAACAAGGGCATTGATGATG | 33 |
| | AS2 | | | | |
| chr8 | RP11-29A2.1 | 5296567 | 5296629 | TGACAAAGTAATAGATGATG | 34 |
| chr13 | DACH1 | 71425420 | 71425482 | AGACAAAGGCAGAGATGAAG | 35 |
| chr4 | TRMT112P1 | 116376959 | 116377021 | CTACAAAGGCATATGTGATG | 36 |
| chr19 | CTD- | 16616586 | 16616648 | TGACAAAGGCAGAGATGAAG | 37 |
| | 3222D19.4 | | | | |
| chr12 | SLC11A2 | 50995656 | 50995718 | CAACAAAGACATTGATGATG | 38 |
| chr19 | CEP89 | 32959414 | 32959476 | CAACAAAGGAAAAGATGATG | 39 |
| chrX | ZDHHC15 | 75427692 | 75427754 | GGACAAAGGAAGAGATGATG | 40 |
| chr10 | RP11- | 33614046 | 33614108 | CGATAAAGCAATAGATGATG | 41 |
| | 476F14.1 | | | | |

TB7 Off-Targets

| | | | | | SEQ |
|---|---|---|---|---|---|
| | | | | | ID |
| Chromosome | Gene Name | start | end | Sequence | NO |
| chr1 | NA | 191016245 | 191016307 | AGTTTTCACCAGTTCCACTG | 42 |
| chr3 | AC117507.1 | 188235173 | 188235235 | AGTTTGCACCCGTTCCACTG | 43 |

TB48 Off-Target

| | | | | | SEQ |
|---|---|---|---|---|---|
| | | | | | ID |
| Chromosome | Gene Name | start | end | Sequence | NO |
| chr10 | ADAM12 | 126309067 | 126309129 | CCATGTGTCCACTCTTGGCA | 44 |
| chr11 | TENM4 | 79267265 | 79267327 | CCATATGTCTACTCTTGACA | 45 |
| chr17 | PRKARIA | 68529541 | 68529603 | CAAAGTCTCAACTCTTGTCA | 46 |
| chr7 | AC007652.1 | 35529882 | 35529944 | CACTGGGTCAACTCTTGAGA | 47 |
| chr10 | LINC01435 | 108245291 | 108245353 | CTATGTGTTAACTCCTGACA | 48 |
| chr13 | CTAGE11P | 75201141 | 75201203 | CAATGTGACAACTCATGCCA | 49 |
| chr5 | LINC01194 | 12801104 | 12801166 | CTATGTTTCAACTCCTGACA | 50 |
| chr3 | ZNF589 | 48263035 | 48263097 | CAATATGTAATCTCTTGACA | 51 |
| chr5 | CTD- | 32668382 | 32668444 | CTATGTGTTAACTCTTGACA | 52 |
| | 2004A9.1 | | | | |
| chrX | RP1-52D1.1 | 87340076 | 87340138 | CAAGGTGTCAACTTCTGACA | 53 |
| chr12 | DDX47 | 12823359 | 12823421 | AAATGTGTCAACTCTTGATT | 54 |
| chr8 | C8orf34 | 68660849 | 68660911 | CTATGTGTTAACTCCTGACA | 55 |
| chrX | NAPIL4P2 | 106451667 | 106451729 | CAATGTGGTAACTCTTGTCA | 56 |
| chr3 | CMC1 | 28142472 | 28142534 | AAATGTGTGAACTCTTGACC | 57 |
| chr12 | RP11- | 23276517 | 23276579 | GAATGTGTAAACTCTTGACC | 58 |
| | 153K16.2 | | | | |
| chr7 | RP11- | 116187375 | 116187437 | CAGTGTGTCGACTCTTGAAA | 59 |
| | 667F14.1 | | | | |
| chr14 | PRKD1 | 29607000 | 29607062 | TAATGTGTCAACTCATAACA | 60 |
| chr1 | FAF1 | 50529073 | 50529135 | TAGGGTGTCAACTCTTGACA | 61 |
| chr3 | PLSCR2 | 146458041 | 146458103 | CAATGTAACAACTCTTTACA | 62 |
| chr4 | PCDH7 | 30917027 | 30917089 | CAGAGTGTCAACTCCTGACA | 63 |
| chr7 | EEF1A1P27 | 20580549 | 20580611 | CAATATGTGAACTCTTGTCA | 64 |
| chr3 | RP11- | 96248457 | 96248519 | CAATGGGTCAGCTTTTGACA | 65 |
| | 598F17.2 | | | | |

TB8-Off-Targets

| | | | | | SEQ |
|---|---|---|---|---|---|
| | | | | | ID |
| Chromosome | Gene Name | start | end | Sequence | NO |
| chr16 | GPR139 | 20074350 | 20074412 | GAAGCTGAGCTTGCTGGCTC | 66 |
| chr1 | RGSL1 | 182554800 | 182554862 | GAAACTGAGCTTGCTGCCTC | 67 |
| chr18 | NA | 66227816 | 66227878 | TATACTGATCTTGCTAGCTC | 68 |
| chr6 | C6orf163 | 87354895 | 87354957 | TAAACAGAGCTGGCTCTCTC | 69 |
| chr20 | SUMO1P1 | 53902185 | 53902247 | TAAACTGTGCTTACTCGCTC | 70 |
| chr5 | NA | 99737598 | 99737660 | TAAAATGAGCCTGCTCTCTC | 71 |
| chr5 | CTD- | 99574606 | 99574668 | TAAAATGAGCCTGCTCTCTC | 72 |
| | 2151A2.1 | | | | |

As demonstrated in FIGS. 12C-D, off-target editing events were rare for all 4 gRNAs (FIG. 12C-D), with a single instance of off-target editing observed with gRNA TB8, where edited cells (orange bars) exceeded an indel threshold set at 0.1%, which was not observed in matched, mock edited cells (blue bars, FIG. 12C). Based on high efficiency editing of primary human CD34⁺ HSCs and rare off-target editing events, these four gRNAs were taken forward to evaluate the potential for highly efficient ccr5 gene editing to generate HIV refractory immune cells.

### Example 7

### Disruption of Ccr5 Gene in Human T Cells Induced by Exemplary gRNAs

This example shows four optimal CCR5-specific gRNAs achieved high-efficiency editing of ccr5 gene at the genetic level in CD34P HSCs.

Human PBMCs were stimulated with phytohemagglutinin (PHA) for 3 days, electroporated with either a non-specific gRNA targeting GFP (green), single gRNAs targeting CCR5 (TB7-brown, TB8-pink, TB48-blue, TB50-red), or a dual gRNA approach (purple). The guides TB48 and TB50 were tested as a dual guide approach to recapitulate the deletion of the gene regions flanking the naturally occurring Δ32 deletion. 48-hours post electroporation, gene disruption was measured by Sanger sequencing of ccr5 amplified from genomic DNA. Table 5 provides CCR5 gRNAs, target sequences and TIDE primer sequences used in this example.

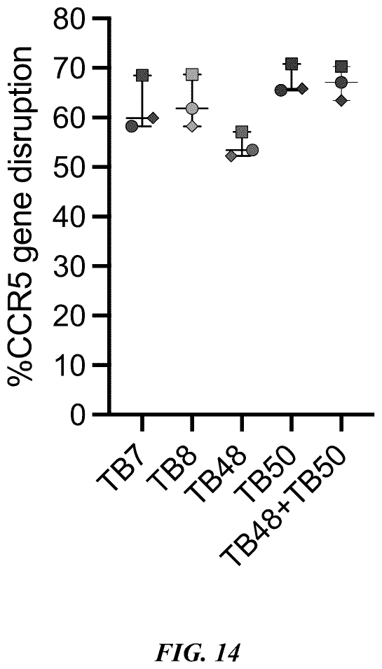

FIG. 14 shows that the four optimal gRNAs and dual guide approach induced robust editing in primary human T cells, with gene disruption percentages ranging from 52% to 70% (FIG. 14).

TABLE 5
Exemplary CCR5 gRNAs, target sequences and TIDE primer sequences.

| | | Location | | | | |
|---|---|---|---|---|---|---|
| CCR5 | | Relative | Forward | Forward | Reverse | Reverse |
| Guide | Target | to Exon 3 | TIDE Primer | TIDE Primer | TIDE Primer | TIDE Primer |
| Names | Sequence | (ORF) | Name | Sequence | Name | Sequence |
| TB48 | CAATGTGT | ORF | PP5 | TTATACATC | PP5 (44/48) | TTTCGACAC |
| | CAACTCTT | | (44/48) F | GGAGCCCTG | R | CGAAGCAG |
| | GACA (SEQ | | | CC (SEQ ID | | AGT (SEQ ID |
| | ID NO: 11) | | | NO: 15) | | NO: 16) |
| TB50 | CGACAAA | ORF | Amp3_ | GCTGTGAGG | Amp3_650 | TCGGAGTG |
| | GGCATAG | | 650 F | CTTATCTTC | R | AAGGGAGA |
| | ATGATG | | | ACCATC | | GTTTGTC |
| | (SEQ ID | | | (SEQ ID NO: | | (SEQ ID NO: |
| | NO: 12) | | | 17) | | 18) |
| TB8 | TAAACTG | ORF | Amp3_ | GCTGTGAGG | Amp3_650 | TCGGAGTG |
| | AGCTTGCT | | 650 F | CTTATCTTC | R | AAGGGAGA |
| | CGCTC | | | ACCATC | | GTTTGTC |
| | (SEQ ID | | | (SEQ ID NO: | | (SEQ ID NO: |
| | NO: 13) | | | 19) | | 20) |
| TB7 | AGTTTACA | ORF | Amp3_ | GCTGTGAGG | Amp3_650 | TCGGAGTG |
| | CCCGATCC | | 650 F | CTTATCTTC | R | AAGGGAGA |
| | ACTG (SEQ | | | ACCATC | | GTTTGTC |
| | ID NO: 10) | | | (SEQ ID NO: | | (SEQ ID NO: |
| | | | | 21) | | 22) |

### Example 8

### High-Efficiency Editing of Human CD34⁺ HSCs does not Limit Lineage Potential

This example evaluates whether high-efficiency editing of human CD34⁺ HSCs affects lineage development.

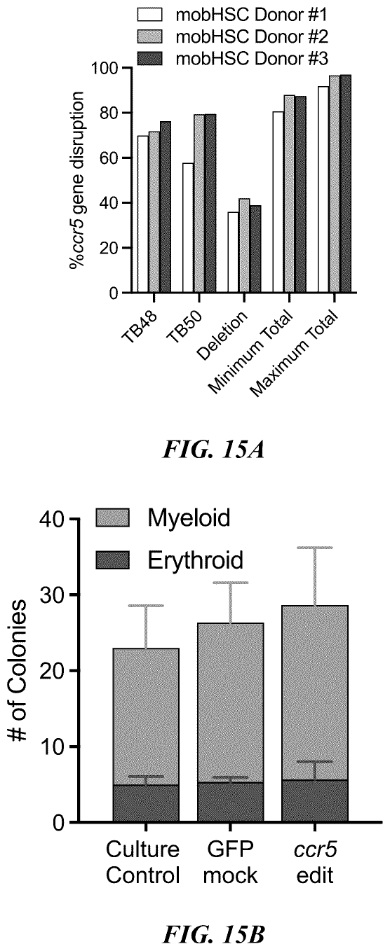

In the pursuit of generating HIV refractory immune systems, mobilized HSCs from healthy adult donors were electroporated with the two most effective gRNAs (TB348 and TB50) complexed with Cas9. Two days after electroporation, editing frequencies of each single gRNA as well as large deletions generated from synchronous dual gRNA editing were measured in genomic DNA, with maximum total ccr5 disruption calculated to range between 91 to 97% in all 3 donors (FIG. 15A). To address whether high efficiency editing of human HSCs affects lineage development, HSCs edited with the dual gRNA approach were assessed for colony formation and lineage potential in a single cell methylcellulose assay. There were no significant differences in the proportion of lineages or absolute number of colonies between mock edited and ccr5-edited HSCs (FIG. 15B).

### Example 9

### In Vivo HSC Editing for Treating β-Thalassemia and Sickle Cell Disease

This example evaluates the BCL11A editing efficiency of several guide RNAs targeting the erythroid specific enhancer of the BCL11A gene in HSCs, particularly erythroid DNase I hypersensitive sites (DHS) +55 and +58 of the BCL11A gene.

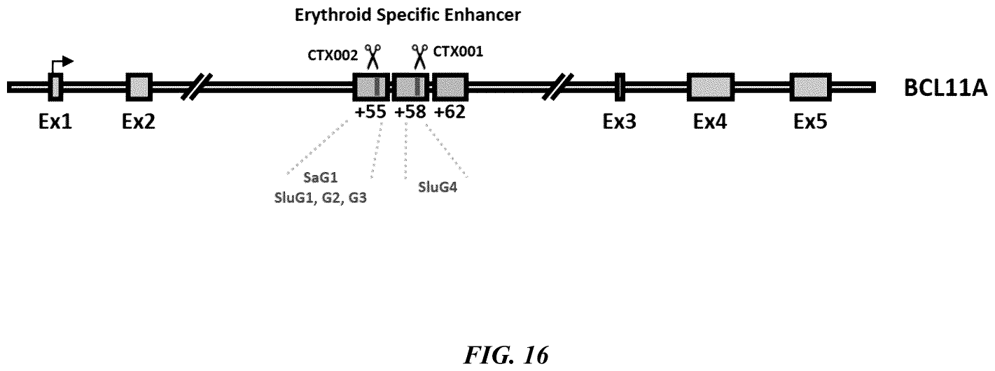

FIG. 16 illustrates a BCL11A gene and locations of gRNA binding sites in the erythroid specific enhancer of the BCL11A gene. The gRNAs selected for editing of the BCL11A gene include SaG1, SluG1, SluG2, SluG3, which target DHS +55 and SluG4 targeting DHS +58.

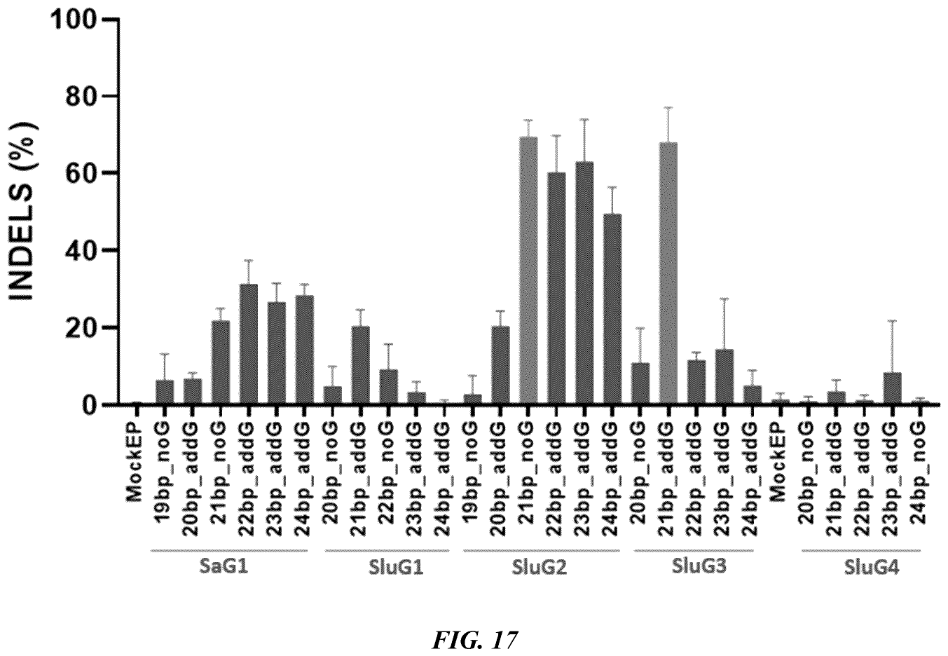

FIG. 17 is a plot showing gene editing efficiency by guide RNAs SaG1, SluG1, SluG2, SluG3 and SluG4. The two guide RNAs (SluG2 and SluG3) comprising a 21 bp spacer sequence targeting DHS +55 were selected for AAV mediated in vivo editing of HSC.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/of” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for in vivo editing hematopoietic stem cells (HSCs) and/or hematopoietic progenitor cells (HPCs) in a subject in need thereof, comprising
administering to the subject a mobilization composition capable of mobilizing the HSCs and/or HPCs in the subject; and
administering to the subject a plurality of adeno-associated virus 9 (AAV9) vectors encapsulating (a) at least one guide RNA (gRNA) that targets a genomic region of interest or a nucleic acid encoding the at least one gRNA, and (b) a nucleic acid encoding a RNA-guided endonuclease, thereby editing the HSCs and/or HPCs in the subject.

2. The method of claim 1, further comprising administering to the subject a second mobilization composition capable of mobilizing HSC and/or HPC in the subject after administering a first mobilization composition to the subject.

3. The method of claim 2, wherein the subject is administered with the first mobilization composition and/or the second mobilization composition daily for two, three, four, five, six, seven, or eight consecutive days.

4. The method of any one of claims 2-3, wherein the first mobilization composition is different from the second mobilization composition.

5. The method of any one of claims 2-4, wherein the first mobilization composition is administered to the subject about one hour to about six hours before the administration of the second mobilization composition.

6. The method of any one of claims 1-5, wherein the first mobilization composition and/or the second mobilization composition comprises a mobilization agent selected from the group consisting of plerixafor or an analog or derivative thereof, granulocyte colony-stimulating factor (G-CSF) or an analog or derivatives thereof, GRO-0 or an analog or derivative thereof, granulocyte macrophage colony stimulating factor (GM-CSF) or an analog or derivative thereof, stem cell factor or an analog or derivative thereof, a modulator of SDF-1/CXCR4 axis, a sphingosine-1-phosphate (S1P) agonist, a VCAM/VLA4 inhibitor, parathyroid hormone (PTH) or an analog or derivative thereof, a proteosome inhibitor, and a combination thereof.

7. The method of claim 6, wherein the mobilization agent is administered to the subject in an amount of about 0.1-20 mg/kg of the subject per administration.

8. The method of claims 2-7, wherein the first mobilization composition comprises plerixafor, and the second mobilization composition comprises plerixafor and G-CSF.

9. The method of any one of claims 1-8, wherein the subject is administered with the plurality of AAV9 vectors once, two times, or three times.

10. The method of any one of claims 1-9, wherein the plurality of AAV9 vectors is administered to the subject after the administration of the first and/or the second mobilization composition, and optionally at least about 0.5 hour, 1 hour, 1.5 hours, 2 hours, 3 hours, or 4 hours after the administration of the first and/or the second mobilization composition.

11. The method of any one of claims 1-10, wherein the plurality of AAVs is administered to the subject at a dose of about 1E14 v/kg to 5E14 vg/kg per administration.

12. The method of any one of claims 1-11, comprising identifying a subject in need of the administration.

13. The method of any one of claims 1-12, wherein the genomic region of interest comprises a gene of interest.

14. The method of claim 13, wherein the gene of interest is B-cell lymphoma/leukemia 11A (BCL11A) gene and the subject is a subject having a β-thalassemia and sickle cell disease.

15. The method of claim 14, wherein at least one of the plurality of AAV9 vectors comprises a nucleic acid encoding the RNA-guided endonuclease and the gRNA that targets BCL11A gene.

16. The method of any one of claims 1-15, wherein the nucleic acid encoding a RNA-guided endonuclease is a mRNA of the RNA-guided endonuclease.

17. The method of any one of claims 1-16, wherein the RNA-guided endonuclease is a Cas9 endonuclease.

18. The method of claim 17, wherein the Cas9 endonuclease is S. pyogenes Cas9, S. aureus Cas9, N. meningitides Cas9, S. thermophilus Cas9, S. thermophilus 3 Cas9, T. denticola Cas9, or a variant thereof.

19. The method of any one of claims 1-18, wherein the at least one gRNA is a single-guide RNA (sgRNA).

20. The method of any one of claims 1-19, wherein (a) the at least one gRNA or the nucleic acid encoding the at least one gRNA, and (b) the nucleic acid encoding the RNA-guided nuclease are encapsulated in a same AAV9.

21. The method of any one of claims 1-19, wherein (a) the at least one gRNA or the nucleic acid encoding the at least one gRNA, and (b) the nucleic acid encoding the RNA-guided nuclease are encapsulated in separate AAV9 vectors.

22. The method of any one of claims 1-21, wherein the plurality of AAV9 vectors are HSC-tropic.

23. The method of any one of claims 1-22, wherein the subject is human.

24. The method of any one of claims 1-23, wherein the plurality of AAV9 vectors is administered to the subject via intravenous administration or systemic administration.

25. An adeno-associated virus (AAV) vector, comprising an AAV9 capsid encapsulating (a) one or two guide RNAs (gRNAs) that targets a genomic region of interest, or a nucleic acid encoding the one or two gRNAs; and (b) a nucleic acid encoding a RNA-guided endonuclease, wherein the genomic region of interest comprises a gene of interest that is preferentially expressed in hematopoietic cells.

26. A composition, comprising a first adeno-associated virus (AAV) vector comprising an AAV9 capsid encapsulating one or two guide RNAs (gRNAs) that target a genomic region of interest or a nucleic acid encoding the one or two gRNAs; and a second AAV vector comprising an AAV9 capsid encapsulating a nucleic acid encoding a RNA-guided endonuclease, wherein the genomic region of interest comprises a gene of interest that is preferentially expressed in hematopoietic cells.

27. The AAV vector of claim 25 or the composition of claim 26, wherein the gene of interest is a gene that is preferentially expressed in hematopoietic stem cells (HSCs) and/or hematopoietic progenitor cells (HPCs).

28. The AAV vector or the composition of any one of claims 25-27, wherein the nucleic acid encoding a RNA-guided endonuclease is a mRNA of the RNA-guided endonuclease.

29. The AAV vector or the composition of any one of claims 25-28, wherein the RNA-guided endonuclease is a Cas9 endonuclease.

30. The AAV vector or the composition of claim 29, wherein the Cas9 endonuclease is S. pyogenes Cas9 (SpCas9), S. aureus Cas9 (SaCas9), S. lugdunensis Cas9 (SluCas9), N. meningitidis Cas9, S. thermophilus Cas9, S. thermophilus 3 Cas9, T. denticola Cas9, C. jejuni Cas9 (CjCas9), or a variant thereof.

31. The AAV vector or the composition of any one of claims 25-30, wherein at least one of the one or two gRNAs is a single-guide RNA (sgRNA).

32. The AAV vector or the composition of any one of claims 25-31, wherein at least one of the one or two gRNAs targets the erythroid specific enhancer of BCL11A gene.

33. A pharmaceutical composition, comprising the AAV vector or the composition of any one of claims 25-32.


SEQUENCE LISTING (72 sequences)

SEQ ID NO: 1 - RNA, 80 nt, synthetic construct
  misc_feature 1..80: note = Synthetic nucleotide
  source 1..80: mol_type = other RNA
gttttagagctagaaatagcaagttaaaataaggctagtccgttatcaacttgaaaaagtggcaccgagtcggtgctttt

SEQ ID NO: 2 - skipped

SEQ ID NO: 3 - RNA, 21 nt, synthetic construct
  source 1..21: mol_type = other RNA
aagccatgtgcacaactctga

SEQ ID NO: 4 - RNA, 21 nt, synthetic construct
  source 1..21: mol_type = other RNA
ctgactgggtcaccagcccac

SEQ ID NO: 5 - RNA, 20 nt, synthetic construct
  source 1..20: mol_type = other RNA
agtttacacccgatccactg

SEQ ID NO: 6 - RNA, 20 nt, synthetic construct
  source 1..20: mol_type = other RNA
caatgtgtcaactcttgaca

SEQ ID NO: 7 - RNA, 20 nt, synthetic construct
  source 1..20: mol_type = other RNA
cgacaaaggcatagatgatg

SEQ ID NO: 8 - DNA, 21 bp, synthetic construct
  source 1..21: mol_type = other DNA
aagccatgtgcacaactctga

SEQ ID NO: 9 - DNA, 21 bp, synthetic construct
  source 1..21: mol_type = other DNA
ctgactgggtcaccagcccac

SEQ ID NO: 10 - DNA, 20 bp, synthetic construct
  source 1..20: mol_type = other DNA
agtttacacccgatccactg

SEQ ID NO: 11 - DNA, 20 bp, synthetic construct
  source 1..20: mol_type = other DNA
caatgtgtcaactcttgaca

SEQ ID NO: 12 - DNA, 20 bp, synthetic construct
  source 1..20: mol_type = other DNA
cgacaaaggcatagatgatg

SEQ ID NO: 13 - DNA, 20 bp, synthetic construct
  source 1..20: mol_type = other DNA
taaactgagcttgctcgctc

SEQ ID NO: 14 - RNA, 20 nt, synthetic construct
  source 1..20: mol_type = other RNA
taaactgagcttgctcgctc

SEQ ID NO: 15 - DNA, 20 bp, synthetic construct
  source 1..20: mol_type = other DNA
ttatacatcggagccctgcc

SEQ ID NO: 16 - DNA, 20 bp, synthetic construct
  source 1..20: mol_type = other DNA
tttcgacaccgaagcagagt

SEQ ID NO: 17 - DNA, 24 bp, synthetic construct
  source 1..24: mol_type = other DNA
gctgtgaggcttatcttcaccatc

SEQ ID NO: 18 - DNA, 23 bp, synthetic construct
  source 1..23: mol_type = other DNA
tcggagtgaagggagagtttgtc

SEQ ID NO: 19 - DNA, 24 bp, synthetic construct
  source 1..24: mol_type = other DNA
gctgtgaggcttatcttcaccatc

SEQ ID NO: 20 - DNA, 23 bp, synthetic construct
  source 1..23: mol_type = other DNA
tcggagtgaagggagagtttgtc

SEQ ID NO: 21 - DNA, 24 bp, synthetic construct
  source 1..24: mol_type = other DNA
gctgtgaggcttatcttcaccatc

SEQ ID NO: 22 - DNA, 23 bp, synthetic construct
  source 1..23: mol_type = other DNA
tcggagtgaagggagagtttgtc

SEQ ID NO: 23 - DNA, 20 bp, Homo sapiens
  source 1..20: mol_type = genomic DNA
ctacaagggaatagatgatg

SEQ ID NO: 24 - DNA, 20 bp, Homo sapiens
  source 1..20: mol_type = genomic DNA
ctacaaaggcataaatgaag

SEQ ID NO: 25 - DNA, 20 bp, Homo sapiens
  source 1..20: mol_type = genomic DNA
ctacaaaggcatagttcatg

SEQ ID NO: 26 - DNA, 20 bp, Homo sapiens
  source 1..20: mol_type = genomic DNA
agacaaaggcctcgatgatg

SEQ ID NO: 27 - DNA, 20 bp, Homo sapiens
  source 1..20: mol_type = genomic DNA
agacagaggcttagatgatg

SEQ ID NO: 28 - DNA, 20 bp, Homo sapiens
  source 1..20: mol_type = genomic DNA
cgaaaatggtatagatgatg

SEQ ID NO: 29 - DNA, 20 bp, Homo sapiens
  source 1..20: mol_type = genomic DNA
agacaaagccacagatgatg

SEQ ID NO: 30 - DNA, 20 bp, Homo sapiens
  source 1..20: mol_type = genomic DNA
tgacacaggaatagatgatg

SEQ ID NO: 31 - DNA, 20 bp, Homo sapiens
  source 1..20: mol_type = genomic DNA
cgatgaaggcgtagatgatg

SEQ ID NO: 32 - DNA, 20 bp, Homo sapiens
  source 1..20: mol_type = genomic DNA
cgaaaaaggcatagaggaag

SEQ ID NO: 33 - DNA, 20 bp, Homo sapiens
  source 1..20: mol_type = genomic DNA
caacaagggcattgatgatg

SEQ ID NO: 34 - DNA, 20 bp, Homo sapiens
  source 1..20: mol_type = genomic DNA
tgacaaagtaatagatgatg

SEQ ID NO: 35 - DNA, 20 bp, Homo sapiens
  source 1..20: mol_type = genomic DNA
agacaaaggcagagatgaag

SEQ ID NO: 36 - DNA, 20 bp, Homo sapiens
  source 1..20: mol_type = genomic DNA
ctacaaaggcatatgtgatg

SEQ ID NO: 37 - DNA, 20 bp, Homo sapiens
  source 1..20: mol_type = genomic DNA
tgacaaaggcagagatgaag

SEQ ID NO: 38 - DNA, 20 bp, Homo sapiens
  source 1..20: mol_type = genomic DNA
caacaaagacattgatgatg

SEQ ID NO: 39 - DNA, 20 bp, Homo sapiens
  source 1..20: mol_type = genomic DNA
caacaaaggaaaagatgatg

SEQ ID NO: 40 - DNA, 20 bp, Homo sapiens
  source 1..20: mol_type = genomic DNA
ggacaaaggaagagatgatg

SEQ ID NO: 41 - DNA, 20 bp, Homo sapiens
  source 1..20: mol_type = genomic DNA
cgataaagcaatagatgatg

SEQ ID NO: 42 - DNA, 20 bp, Homo sapiens
  source 1..20: mol_type = genomic DNA
agttttcaccagttccactg

SEQ ID NO: 43 - DNA, 20 bp, Homo sapiens
  source 1..20: mol_type = genomic DNA
agtttgcacccgttccactg

SEQ ID NO: 44 - DNA, 20 bp, Homo sapiens
  source 1..20: mol_type = genomic DNA
ccatgtgtccactcttggca

SEQ ID NO: 45 - DNA, 20 bp, Homo sapiens
  source 1..20: mol_type = genomic DNA
ccatatgtctactcttgaca

SEQ ID NO: 46 - DNA, 20 bp, Homo sapiens
  source 1..20: mol_type = genomic DNA
caaagtctcaactcttgtca

SEQ ID NO: 47 - DNA, 20 bp, Homo sapiens
  source 1..20: mol_type = genomic DNA
cactgggtcaactcttgaga

SEQ ID NO: 48 - DNA, 20 bp, Homo sapiens
  source 1..20: mol_type = genomic DNA
ctatgtgttaactcctgaca

SEQ ID NO: 49 - DNA, 20 bp, Homo sapiens
  source 1..20: mol_type = genomic DNA
caatgtgacaactcatgcca

SEQ ID NO: 50 - DNA, 20 bp, Homo sapiens
  source 1..20: mol_type = genomic DNA
ctatgtttcaactcctgaca

SEQ ID NO: 51 - DNA, 20 bp, Homo sapiens
  source 1..20: mol_type = genomic DNA
caatatgtaatctcttgaca

SEQ ID NO: 52 - DNA, 20 bp, Homo sapiens
  source 1..20: mol_type = genomic DNA
ctatgtgttaactcttgaca

SEQ ID NO: 53 - DNA, 20 bp, Homo sapiens
  source 1..20: mol_type = genomic DNA
caaggtgtcaacttctgaca

SEQ ID NO: 54 - DNA, 20 bp, Homo sapiens
  source 1..20: mol_type = genomic DNA
aaatgtgtcaactcttgatt

SEQ ID NO: 55 - DNA, 20 bp, Homo sapiens
  source 1..20: mol_type = genomic DNA
ctatgtgttaactcctgaca

SEQ ID NO: 56 - DNA, 20 bp, Homo sapiens
  source 1..20: mol_type = genomic DNA
caatgtggtaactcttgtca

SEQ ID NO: 57 - DNA, 20 bp, Homo sapiens
  source 1..20: mol_type = genomic DNA
aaatgtgtgaactcttgacc

SEQ ID NO: 58 - DNA, 20 bp, Homo sapiens
  source 1..20: mol_type = genomic DNA
gaatgtgtaaactcttgacc

SEQ ID NO: 59 - DNA, 20 bp, Homo sapiens
  source 1..20: mol_type = genomic DNA
cagtgtgtcgactcttgaaa

SEQ ID NO: 60 - DNA, 20 bp, Homo sapiens
  source 1..20: mol_type = genomic DNA
taatgtgtcaactcataaca

SEQ ID NO: 61 - DNA, 20 bp, Homo sapiens
  source 1..20: mol_type = genomic DNA
tagggtgtcaactcttgaca

SEQ ID NO: 62 - DNA, 20 bp, Homo sapiens
  source 1..20: mol_type = genomic DNA
caatgtaacaactctttaca

SEQ ID NO: 63 - DNA, 20 bp, Homo sapiens
  source 1..20: mol_type = genomic DNA
cagagtgtcaactcctgaca

SEQ ID NO: 64 - DNA, 20 bp, Homo sapiens
  source 1..20: mol_type = genomic DNA
caatatgtgaactcttgtca

SEQ ID NO: 65 - DNA, 20 bp, Homo sapiens
  source 1..20: mol_type = genomic DNA
caatgggtcagcttttgaca

SEQ ID NO: 66 - DNA, 20 bp, Homo sapiens
  source 1..20: mol_type = genomic DNA
gaagctgagcttgctggctc

SEQ ID NO: 67 - DNA, 20 bp, Homo sapiens
  source 1..20: mol_type = genomic DNA
gaaactgagcttgctgcctc

SEQ ID NO: 68 - DNA, 20 bp, Homo sapiens
  source 1..20: mol_type = genomic DNA
tatactgatcttgctagctc

SEQ ID NO: 69 - DNA, 20 bp, Homo sapiens
  source 1..20: mol_type = genomic DNA
taaacagagctggctctctc

SEQ ID NO: 70 - DNA, 20 bp, Homo sapiens
  source 1..20: mol_type = genomic DNA
taaactgtgcttactcgctc

SEQ ID NO: 71 - DNA, 20 bp, Homo sapiens
  source 1..20: mol_type = genomic DNA
taaaatgagcctgctctctc

SEQ ID NO: 72 - DNA, 20 bp, Homo sapiens
  source 1..20: mol_type = genomic DNA
taaaatgagcctgctctctc